SEQUENCE LISTING (10 sequences)

SEQ ID NO: 1 - DNA, 3285 bp, Artificial Sequence
  cDNA encoding isoform 1 of human PLC-L1
atggccgagggcgcggccggcagggaggatccggcgccgcccgacgcggcggggggcgaagacgacccccgagtgggcccggatgccgccggggactgcgtgacggcggcctctgggggccggatgagggaccgtcgcagcggggtcgcactgccaggcgccgcggggaccccagcggacagcgaggcgggcctcctggaggcagcacgggcgaccccccggcgcagcagcatcatcaaggatccttcaaaccaaaaatgtggtggaagaaagaaaaccgtgtctttcagcagcatgccatcggaaaagaaaattagcagtgcaaatgactgcatcagcttcatgcaagctggctgtgagttgaagaaagtccggccaaattctcgcatttacaaccgttttttcactctggacacagaccttcaagctcttcgctgggaaccttcaaagaaagacctcgagaaagccaagcttgatatttctgccataaaagagatcagactggggaaaaacacggaaacatttagaaacaatggccttgctgaccagatctgtgaggactgtgccttttccatactccacggggaaaactatgagtctctggacctagttgccaattcagcagatgtggcaaacatctgggtgtctgggttacggtacctggtttctcgaagtaagcagcctcttgattttatggagggcaaccagaacacaccacggttcatgtggttgaaaacagtgtttgaagcagcagatgttgatgggaatgggattatgttggaagacacctctgtagagttaataaaacaactcaaccctactctgaaggaagccaagatcaggttaaagtttaaagaaatccagaagagcaaggaaaaactaaccacccgcgtgaccgaagaggaattttgtgaagctttttgtgaactttgcaccaggccagaagtgtatttcttacttgtacagatatctaaaaacaaagaatatttggatgccaatgatctcatgctctttttagaagctgagcaaggagtcacccatatcaccgaggatatatgcttagacatcataaggagatacgaactttctgaagagggacgtcaaaaagggtttcttgcaattgatggctttacccagtatttattgtcatcagaatgtgacatttttgatcctgagcaaaagaaggttgcccaagatatgacccagccattatctcactactatatcaatgcctctcataacacctatctaatagaagaccagttcagggggccagctgacatcaatgggtacattagagctttgaaaatgggctgtcgaagcgttgaactcgatgtaagtgatggttcagataatgaaccaatcctttgtaatcgaaataacatgacaacccatgtttcctttcgaagtgtcatagaggtaataaataaatttgcctttgttgcttctgaatacccactcattctttgcttgggaaatcactgctccttgccgcagcagaaggtaatggctcaacagatgaaaaaggtctttggcaataaactctatactgaagcacctttgccctcagaatcctacctcccatcaccagaaaaattaaaaagaatgatcattgtgaaaggaaagaagttgccttctgatccagatgtgttagaaggagaagtaacagatgaagatgaagaagctgaaatgtctcgaaggatgtcggtagattacaatggtgagcagaagcaaatccgactctgtagggagctctctgatttggtgtctatttgtaaatctgttcaatacagggattttgaactatctatgaaaagccaaaactattgggaaatgtgttcatttagtgaaacagaggccagccgcattgcaaatgagtacccagaggattttgttaattataataagaagttcttatcaagaatctatccaagtgccatgaggatcgattccagtaacttgaatccacaggacttttggaattgtggctgtcagattgtagcaatgaattttcagactccgggtccaatgatggaccttcacacgggctggtttcttcaaaacgggggatgtggttatgttctaaggccgtctataatgcgagatgaagtttcttacttcagcgcaaatacaaagggcattctacctggggtgtctcctctagctcttcatatcaagatcatcagtggtcagaatttcccaaagcccaagggagcttgtgccaaaggggatgtcatagatccctatgtttgtatagagatacacggaattccagcggattgttcggaacaaagaactaaaactgtacagcaaaacagtgataatcctatttttgatgaaacttttgagttccaagtaaacctacctgagctggccatgatccgttttgttgttctggatgatgactacattggggatgagtttatagggcaatatacgataccatttgaatgtttgcagcctggatatcggcatgttcccctgcgttcttttgtgggtgacatcatggagcacgtaaccctttttgtccacatagcaataactaatcgaagtggaggaggaaaggcacagaagcgcagtctttcagtgagaatggggaagaaagttcgggaatataccatgctcaggaatatcggtcttaaaaccattgatgacatctttaaaatagcggttcatccattacgagaagccatagatatgagagaaaatatgcagaatgcaatcgtgtctattaaggaactatgtggactccctccaattgccagtctgaagcagtgcctgttaactctgtcatctcggctcatcaccagtgacaatactccttcagtctcacttgtgatgaaagacagctttccttacctggagcctctgggtgcaattccagatgtgcagaaaaagatgctgactgcttatgatctgatgattcaagagagccggtttctcatagaaatggcggacacagtccaggaaaagattgtacagtgtcagaaagcagggatggagttccatgaagaacttcataatttgggggcaaaagaaggcttgaagggaagaaaactcaacaaagcaactgagagctttgcttggaacattacagtattgaagggccaaggagatctgttgaagaatgccaagaatgaagctatagaaaacatgaagcagatccagctggcatgcctgtcctgtggactgagtaaagcccccagcagcagtgctgaggccaagagcaagcgcagcctggaagccatagaggagaaggaaagtagtgaggagaatgggaagctg

SEQ ID NO: 2 - DNA, 2991 bp, Artificial Sequence
  cDNA encoding isoform 2 of human PLC-L1
atgccatcggaaaagaaaattagcagtgcaaatgactgcatcagcttcatgcaagctggctgtgagttgaagaaagtccggccaaattctcgcatttacaaccgttttttcactctggacacagaccttcaagctcttcgctgggaaccttcaaagaaagacctcgagaaagccaagcttgatatttctgccataaaagagatcagactggggaaaaacacggaaacatttagaaacaatggccttgctgaccagatctgtgaggactgtgccttttccatactccacggggaaaactatgagtctctggacctagttgccaattcagcagatgtggcaaacatctgggtgtctgggttacggtacctggtttctcgaagtaagcagcctcttgattttatggagggcaaccagaacacaccacggttcatgtggttgaaaacagtgtttgaagcagcagatgttgatgggaatgggattatgttggaagacacctctgtagagttaataaaacaactcaaccctactctgaaggaagccaagatcaggttaaagtttaaagaaatccagaagagcaaggaaaaactaaccacccgcgtgaccgaagaggaattttgtgaagctttttgtgaactttgcaccaggccagaagtgtatttcttacttgtacagatatctaaaaacaaagaatatttggatgccaatgatctcatgctctttttagaagctgagcaaggagtcacccatatcaccgaggatatatgcttagacatcataaggagatacgaactttctgaagagggacgtcaaaaagggtttcttgcaattgatggctttacccagtatttattgtcatcagaatgtgacatttttgatcctgagcaaaagaaggttgcccaagatatgacccagccattatctcactactatatcaatgcctctcataacacctatctaatagaagaccagttcagggggccagctgacatcaatgggtacattagagctttgaaaatgggctgtcgaagcgttgaactcgatgtaagtgatggttcagataatgaaccaatcctttgtaatcgaaataacatgacaacccatgtttcctttcgaagtgtcatagaggtaataaataaatttgcctttgttgcttctgaatacccactcattctttgcttgggaaatcactgctccttgccgcagcagaaggtaatggctcaacagatgaaaaaggtctttggcaataaactctatactgaagcacctttgccctcagaatcctacctcccatcaccagaaaaattaaaaagaatgatcattgtgaaaggaaagaagttgccttctgatccagatgtgttagaaggagaagtaacagatgaagatgaagaagctgaaatgtctcgaaggatgtcggtagattacaatggtgagcagaagcaaatccgactctgtagggagctctctgatttggtgtctatttgtaaatctgttcaatacagggattttgaactatctatgaaaagccaaaactattgggaaatgtgttcatttagtgaaacagaggccagccgcattgcaaatgagtacccagaggattttgttaattataataagaagttcttatcaagaatctatccaagtgccatgaggatcgattccagtaacttgaatccacaggacttttggaattgtggctgtcagattgtagcaatgaattttcagactccgggtccaatgatggaccttcacacgggctggtttcttcaaaacgggggatgtggttatgttctaaggccgtctataatgcgagatgaagtttcttacttcagcgcaaatacaaagggcattctacctggggtgtctcctctagctcttcatatcaagatcatcagtggtcagaatttcccaaagcccaagggagcttgtgccaaaggggatgtcatagatccctatgtttgtatagagatacacggaattccagcggattgttcggaacaaagaactaaaactgtacagcaaaacagtgataatcctatttttgatgaaacttttgagttccaagtaaacctacctgagctggccatgatccgttttgttgttctggatgatgactacattggggatgagtttatagggcaatatacgataccatttgaatgtttgcagcctggatatcggcatgttcccctgcgttcttttgtgggtgacatcatggagcacgtaaccctttttgtccacatagcaataactaatcgaagtggaggaggaaaggcacagaagcgcagtctttcagtgagaatggggaagaaagttcgggaatataccatgctcaggaatatcggtcttaaaaccattgatgacatctttaaaatagcggttcatccattacgagaagccatagatatgagagaaaatatgcagaatgcaatcgtgtctattaaggaactatgtggactccctccaattgccagtctgaagcagtgcctgttaactctgtcatctcggctcatcaccagtgacaatactccttcagtctcacttgtgatgaaagacagctttccttacctggagcctctgggtgcaattccagatgtgcagaaaaagatgctgactgcttatgatctgatgattcaagagagccggtttctcatagaaatggcggacacagtccaggaaaagattgtacagtgtcagaaagcagggatggagttccatgaagaacttcataatttgggggcaaaagaaggcttgaagggaagaaaactcaacaaagcaactgagagctttgcttggaacattacagtattgaagggccaaggagatctgttgaagaatgccaagaatgaagctatagaaaacatgaagcagatccagctggcatgcctgtcctgtggactgagtaaagcccccagcagcagtgctgaggccaagagcaagcgcagcctggaagccatagaggagaaggaaagtagtgaggagaatgggaagctg

SEQ ID NO: 3 - RNA, 3285 nt, Homo sapiens
auggccgagggcgcggccggcagggaggauccggcgccgcccgacgcggcggggggcgaagacgacccccgagugggcccggaugccgccggggacugcgugacggcggccucugggggccggaugagggaccgucgcagcggggucgcacugccaggcgccgcggggaccccagcggacagcgaggcgggccuccuggaggcagcacgggcgaccccccggcgcagcagcaucaucaaggauccuucaaaccaaaaaugugguggaagaaagaaaaccgugucuuucagcagcaugccaucggaaaagaaaauuagcagugcaaaugacugcaucagcuucaugcaagcuggcugugaguugaagaaaguccggccaaauucucgcauuuacaaccguuuuuucacucuggacacagaccuucaagcucuucgcugggaaccuucaaagaaagaccucgagaaagccaagcuugauauuucugccauaaaagagaucagacuggggaaaaacacggaaacauuuagaaacaauggccuugcugaccagaucugugaggacugugccuuuuccauacuccacggggaaaacuaugagucucuggaccuaguugccaauucagcagauguggcaaacaucugggugucuggguuacgguaccugguuucucgaaguaagcagccucuugauuuuauggagggcaaccagaacacaccacgguucaugugguugaaaacaguguuugaagcagcagauguugaugggaaugggauuauguuggaagacaccucuguagaguuaauaaaacaacucaacccuacucugaaggaagccaagaucagguuaaaguuuaaagaaauccagaagagcaaggaaaaacuaaccacccgcgugaccgaagaggaauuuugugaagcuuuuugugaacuuugcaccaggccagaaguguauuucuuacuuguacagauaucuaaaaacaaagaauauuuggaugccaaugaucucaugcucuuuuuagaagcugagcaaggagucacccauaucaccgaggauauaugcuuagacaucauaaggagauacgaacuuucugaagagggacgucaaaaaggguuucuugcaauugauggcuuuacccaguauuuauugucaucagaaugugacauuuuugauccugagcaaaagaagguugcccaagauaugacccagccauuaucucacuacuauaucaaugccucucauaacaccuaucuaauagaagaccaguucagggggccagcugacaucaauggguacauuagagcuuugaaaaugggcugucgaagcguugaacucgauguaagugaugguucagauaaugaaccaauccuuuguaaucgaaauaacaugacaacccauguuuccuuucgaagugucauagagguaauaaauaaauuugccuuuguugcuucugaauacccacucauucuuugcuugggaaaucacugcuccuugccgcagcagaagguaauggcucaacagaugaaaaaggucuuuggcaauaaacucuauacugaagcaccuuugcccucagaauccuaccucccaucaccagaaaaauuaaaaagaaugaucauugugaaaggaaagaaguugccuucugauccagauguguuagaaggagaaguaacagaugaagaugaagaagcugaaaugucucgaaggaugucgguagauuacaauggugagcagaagcaaauccgacucuguagggagcucucugauuuggugucuauuuguaaaucuguucaauacagggauuuugaacuaucuaugaaaagccaaaacuauugggaaauguguucauuuagugaaacagaggccagccgcauugcaaaugaguacccagaggauuuuguuaauuauaauaagaaguucuuaucaagaaucuauccaagugccaugaggaucgauuccaguaacuugaauccacaggacuuuuggaauuguggcugucagauuguagcaaugaauuuucagacuccggguccaaugauggaccuucacacgggcugguuucuucaaaacgggggaugugguuauguucuaaggccgucuauaaugcgagaugaaguuucuuacuucagcgcaaauacaaagggcauucuaccuggggugucuccucuagcucuucauaucaagaucaucaguggucagaauuucccaaagcccaagggagcuugugccaaaggggaugucauagaucccuauguuuguauagagauacacggaauuccagcggauuguucggaacaaagaacuaaaacuguacagcaaaacagugauaauccuauuuuugaugaaacuuuugaguuccaaguaaaccuaccugagcuggccaugauccguuuuguuguucuggaugaugacuacauuggggaugaguuuauagggcaauauacgauaccauuugaauguuugcagccuggauaucggcauguuccccugcguucuuuugugggugacaucauggagcacguaacccuuuuuguccacauagcaauaacuaaucgaaguggaggaggaaaggcacagaagcgcagucuuucagugagaauggggaagaaaguucgggaauauaccaugcucaggaauaucggucuuaaaaccauugaugacaucuuuaaaauagcgguucauccauuacgagaagccauagauaugagagaaaauaugcagaaugcaaucgugucuauuaaggaacuauguggacucccuccaauugccagucugaagcagugccuguuaacucugucaucucggcucaucaccagugacaauacuccuucagucucacuugugaugaaagacagcuuuccuuaccuggagccucugggugcaauuccagaugugcagaaaaagaugcugacugcuuaugaucugaugauucaagagagccgguuucucauagaaauggcggacacaguccaggaaaagauuguacagugucagaaagcagggauggaguuccaugaagaacuucauaauuugggggcaaaagaaggcuugaagggaagaaaacucaacaaagcaacugagagcuuugcuuggaacauuacaguauugaagggccaaggagaucuguugaagaaugccaagaaugaagcuauagaaaacaugaagcagauccagcuggcaugccuguccuguggacugaguaaagcccccagcagcagugcugaggccaagagcaagcgcagccuggaagccauagaggagaaggaaaguagugaggagaaugggaagcug

SEQ ID NO: 4 - RNA, 2991 nt, Homo sapiens
augccaucggaaaagaaaauuagcagugcaaaugacugcaucagcuucaugcaagcuggcugugaguugaagaaaguccggccaaauucucgcauuuacaaccguuuuuucacucuggacacagaccuucaagcucuucgcugggaaccuucaaagaaagaccucgagaaagccaagcuugauauuucugccauaaaagagaucagacuggggaaaaacacggaaacauuuagaaacaauggccuugcugaccagaucugugaggacugugccuuuuccauacuccacggggaaaacuaugagucucuggaccuaguugccaauucagcagauguggcaaacaucugggugucuggguuacgguaccugguuucucgaaguaagcagccucuugauuuuauggagggcaaccagaacacaccacgguucaugugguugaaaacaguguuugaagcagcagauguugaugggaaugggauuauguuggaagacaccucuguagaguuaauaaaacaacucaacccuacucugaaggaagccaagaucagguuaaaguuuaaagaaauccagaagagcaaggaaaaacuaaccacccgcgugaccgaagaggaauuuugugaagcuuuuugugaacuuugcaccaggccagaaguguauuucuuacuuguacagauaucuaaaaacaaagaauauuuggaugccaaugaucucaugcucuuuuuagaagcugagcaaggagucacccauaucaccgaggauauaugcuuagacaucauaaggagauacgaacuuucugaagagggacgucaaaaaggguuucuugcaauugauggcuuuacccaguauuuauugucaucagaaugugacauuuuugauccugagcaaaagaagguugcccaagauaugacccagccauuaucucacuacuauaucaaugccucucauaacaccuaucuaauagaagaccaguucagggggccagcugacaucaauggguacauuagagcuuugaaaaugggcugucgaagcguugaacucgauguaagugaugguucagauaaugaaccaauccuuuguaaucgaaauaacaugacaacccauguuuccuuucgaagugucauagagguaauaaauaaauuugccuuuguugcuucugaauacccacucauucuuugcuugggaaaucacugcuccuugccgcagcagaagguaauggcucaacagaugaaaaaggucuuuggcaauaaacucuauacugaagcaccuuugcccucagaauccuaccucccaucaccagaaaaauuaaaaagaaugaucauugugaaaggaaagaaguugccuucugauccagauguguuagaaggagaaguaacagaugaagaugaagaagcugaaaugucucgaaggaugucgguagauuacaauggugagcagaagcaaauccgacucuguagggagcucucugauuuggugucuauuuguaaaucuguucaauacagggauuuugaacuaucuaugaaaagccaaaacuauugggaaauguguucauuuagugaaacagaggccagccgcauugcaaaugaguacccagaggauuuuguuaauuauaauaagaaguucuuaucaagaaucuauccaagugccaugaggaucgauuccaguaacuugaauccacaggacuuuuggaauuguggcugucagauuguagcaaugaauuuucagacuccggguccaaugauggaccuucacacgggcugguuucuucaaaacgggggaugugguuauguucuaaggccgucuauaaugcgagaugaaguuucuuacuucagcgcaaauacaaagggcauucuaccuggggugucuccucuagcucuucauaucaagaucaucaguggucagaauuucccaaagcccaagggagcuugugccaaaggggaugucauagaucccuauguuuguauagagauacacggaauuccagcggauuguucggaacaaagaacuaaaacuguacagcaaaacagugauaauccuauuuuugaugaaacuuuugaguuccaaguaaaccuaccugagcuggccaugauccguuuuguuguucuggaugaugacuacauuggggaugaguuuauagggcaauauacgauaccauuugaauguuugcagccuggauaucggcauguuccccugcguucuuuugugggugacaucauggagcacguaacccuuuuuguccacauagcaauaacuaaucgaaguggaggaggaaaggcacagaagcgcagucuuucagugagaauggggaagaaaguucgggaauauaccaugcucaggaauaucggucuuaaaaccauugaugacaucuuuaaaauagcgguucauccauuacgagaagccauagauaugagagaaaauaugcagaaugcaaucgugucuauuaaggaacuauguggacucccuccaauugccagucugaagcagugccuguuaacucugucaucucggcucaucaccagugacaauacuccuucagucucacuugugaugaaagacagcuuuccuuaccuggagccucugggugcaauuccagaugugcagaaaaagaugcugacugcuuaugaucugaugauucaagagagccgguuucucauagaaauggcggacacaguccaggaaaagauuguacagugucagaaagcagggauggaguuccaugaagaacuucauaauuugggggcaaaagaaggcuugaagggaagaaaacucaacaaagcaacugagagcuuugcuuggaacauuacaguauugaagggccaaggagaucuguugaagaaugccaagaaugaagcuauagaaaacaugaagcagauccagcuggcaugccuguccuguggacugaguaaagcccccagcagcagugcugaggccaagagcaagcgcagccuggaagccauagaggagaaggaaaguagugaggagaaugggaagcug

SEQ ID NO: 5 - protein, 1095 aa, Homo sapiens
MAEGAAGREDPAPPDAAGGEDDPRVGPDAAGDCVTAASGGRMRDRRSGVALPGAAGTPADSEAGLLEAARATPRRSSIIKDPSNQKCGGRKKTVSFSSMPSEKKISSANDCISFMQAGCELKKVRPNSRIYNRFFTLDTDLQALRWEPSKKDLEKAKLDISAIKEIRLGKNTETFRNNGLADQICEDCAFSILHGENYESLDLVANSADVANIWVSGLRYLVSRSKQPLDFMEGNQNTPRFMWLKTVFEAADVDGNGIMLEDTSVELIKQLNPTLKEAKIRLKFKEIQKSKEKLTTRVTEEEFCEAFCELCTRPEVYFLLVQISKNKEYLDANDLMLFLEAEQGVTHITEDICLDIIRRYELSEEGRQKGFLAIDGFTQYLLSSECDIFDPEQKKVAQDMTQPLSHYYINASHNTYLIEDQFRGPADINGYIRALKMGCRSVELDVSDGSDNEPILCNRNNMTTHVSFRSVIEVINKFAFVASEYPLILCLGNHCSLPQQKVMAQQMKKVFGNKLYTEAPLPSESYLPSPEKLKRMIIVKGKKLPSDPDVLEGEVTDEDEEAEMSRRMSVDYNGEQKQIRLCRELSDLVSICKSVQYRDFELSMKSQNYWEMCSFSETEASRIANEYPEDFVNYNKKFLSRIYPSAMRIDSSNLNPQDFWNCGCQIVAMNFQTPGPMMDLHTGWFLQNGGCGYVLRPSIMRDEVSYFSANTKGILPGVSPLALHIKIISGQNFPKPKGACAKGDVIDPYVCIEIHGIPADCSEQRTKTVQQNSDNPIFDETFEFQVNLPELAMIRFVVLDDDYIGDEFIGQYTIPFECLQPGYRHVPLRSFVGDIMEHVTLFVHIAITNRSGGGKAQKRSLSVRMGKKVREYTMLRNIGLKTIDDIFKIAVHPLREAIDMRENMQNAIVSIKELCGLPPIASLKQCLLTLSSRLITSDNTPSVSLVMKDSFPYLEPLGAIPDVQKKMLTAYDLMIQESRFLIEMADTVQEKIVQCQKAGMEFHEELHNLGAKEGLKGRKLNKATESFAWNITVLKGQGDLLKNAKNEAIENMKQIQLACLSCGLSKAPSSSAEAKSKRSLEAIEEKESSEENGKL

SEQ ID NO: 6 - protein, 997 aa, Homo sapiens
MPSEKKISSANDCISFMQAGCELKKVRPNSRIYNRFFTLDTDLQALRWEPSKKDLEKAKLDISAIKEIRLGKNTETFRNNGLADQICEDCAFSILHGENYESLDLVANSADVANIWVSGLRYLVSRSKQPLDFMEGNQNTPRFMWLKTVFEAADVDGNGIMLEDTSVELIKQLNPTLKEAKIRLKFKEIQKSKEKLTTRVTEEEFCEAFCELCTRPEVYFLLVQISKNKEYLDANDLMLFLEAEQGVTHITEDICLDIIRRYELSEEGRQKGFLAIDGFTQYLLSSECDIFDPEQKKVAQDMTQPLSHYYINASHNTYLIEDQFRGPADINGYIRALKMGCRSVELDVSDGSDNEPILCNRNNMTTHVSFRSVIEVINKFAFVASEYPLILCLGNHCSLPQQKVMAQQMKKVFGNKLYTEAPLPSESYLPSPEKLKRMIIVKGKKLPSDPDVLEGEVTDEDEEAEMSRRMSVDYNGEQKQIRLCRELSDLVSICKSVQYRDFELSMKSQNYWEMCSFSETEASRIANEYPEDFVNYNKKFLSRIYPSAMRIDSSNLNPQDFWNCGCQIVAMNFQTPGPMMDLHTGWFLQNGGCGYVLRPSIMRDEVSYFSANTKGILPGVSPLALHIKIISGQNFPKPKGACAKGDVIDPYVCIEIHGIPADCSEQRTKTVQQNSDNPIFDETFEFQVNLPELAMIRFVVLDDDYIGDEFIGQYTIPFECLQPGYRHVPLRSFVGDIMEHVTLFVHIAITNRSGGGKAQKRSLSVRMGKKVREYTMLRNIGLKTIDDIFKIAVHPLREAIDMRENMQNAIVSIKELCGLPPIASLKQCLLTLSSRLITSDNTPSVSLVMKDSFPYLEPLGAIPDVQKKMLTAYDLMIQESRFLIEMADTVQEKIVQCQKAGMEFHEELHNLGAKEGLKGRKLNKATESFAWNITVLKGQGDLLKNAKNEAIENMKQIQLACLSCGLSKAPSSSAEAKSKRSLEAIEEKESSEENGKL

SEQ ID NO: 7 - RNA, 19 nt, Artificial Sequence
  PLC-L1 siRNA
guagggagcucucugauuu

SEQ ID NO: 8 - RNA, 19 nt, Artificial Sequence
  PLC-L1 siRNA
gaagaaaguucgggaauau

SEQ ID NO: 9 - RNA, 19 nt, Artificial Sequence
  PLC-L1 siRNA
gguaauggcucaacagaug

SEQ ID NO: 10 - RNA, 19 nt, Artificial Sequence
  PLC-L1 siRNA
gcacagaagcgcagucuuu

(19) United States
(12) Patent Application Publication
(10) Pub. No.: US 2016/0161487 A1
(43) Pub. Date: Jun. 9, 2016

(54) CONTROL OF UTERINE CONTRACTIONS

(71) Applicant: THE UNIVERSITY OF WARWICK, Coventry (GB)

(72) Inventors: Paul BRIGHTON, Coventry (GB); Andrew BLANKS, Coventry (GB)

(21) Appl. No.: 14/905,875

(22) PCT Filed: Jul. 18, 2014
(86) PCT No.: PCT/GB2014/052203

§ 371 Date: Jan. 18, 2016

(30) Foreign Application Priority Data
Jul. 18, 2013 (GB) 1312891.3

(51) Int. Cl. G01N 33/573; C12Q 1/68

(52) U.S. Cl. CPC G01N 33/573; C12Q 1/6883; G01N 2333/916; G01N 2800/368

(57) ABSTRACT

The invention is in the field of pregnancy and labour. A protein has been identified which is significantly down-regulated in labour. Measuring amounts of this protein therefore allows determination of whether or not a patient is in labour. Furthermore, inhibiting this protein, or providing this protein, can be used to induce or prevent labour.

### FIELD OF THE INVENTION

The invention relates to a method of determining whether or not a patient is in labour. The invention also concerns methods for inducing labour in a patient, and methods for preventing labour in a patient. Kits for use in such methods are also provided.

### BACKGROUND OF THE INVENTION

Inappropriate myometrial function is a major contributor to pre-term labour. In the UK, 50,000 babies are born pre-term each year (Berridge et al. Neuron 21(1), 13 (1998)) and these account for up to 70% of infant deaths posing a serious clinical problem. Pre-term babies suffer from a high rate of mortality and morbidity, and those babies that manage to survive have an increased incidence of severe handicap. In addition, these circumstances contribute to massive emotional, social and economic costs. Improving therapies for pre-term labour is especially important as the neonatal survival rate and quality of health of babies born prematurely improves dramatically for each week spent in utero beyond 23 weeks gestation. Current tocolytic therapies designed to prevent pre-term labour by targeting the contractile mechanisms of the myometrial smooth muscle (MSM) cell exhibit relatively poor efficacy.

It is generally accepted that prior to the onset of labour in humans the myometrium undergoes a process of “activation” (Challis et al. Endocr Rev 21(5), 514 (2000)) whereby the muscle becomes both more excitable and more susceptible to stimulation by pro-contractile hormones. It has been demonstrated that in correlation with the process of activation many CAP (Contraction Associated Proteins) genes (e.g. OTR, COX-2, CX-43 etc) increase. At the same time resting membrane potential becomes less hyperpolarised (Parkington et al. Am J Obstet Gynecol 181 (6), 1445 (1999)) and inhibitory pathways that stimulate cAMP-PKA begin to decrease (Lopez et al. Adv Exp Med Biol 395, 435 (1995)). Thus the evolution of this process can be viewed simplistically as a shift in balance from low intrinsic excitability and refractoriness to stimulation, to high intrinsic excitability and susceptibility to stimulation.

The physiological corollary to this process in vivo is the manifestation of weak disordered contractions (Braxton-Hicks) in the weeks preceding labour. In primate models this process is well described in terms of a switch from contractures to contractions in a defined pattern in the days preceding delivery (Nathanielsz et al. Reprod Fertil Dev 7 (3), 595 (1995)). The same pattern occurs when monitoring EMG activity in humans with a notable change in activity 72-48 hrs prior to the onset of labour (Garfield et al. Am J Obstet Gynecol 193 (1), 23 (2005)). It is reasonable to assume that this process is evolving slowly (days-weeks) in normal term pregnancy but in subsets of pre-term patients (excluding those due to infection or placental abruption) this process occurs, for whatever reason, too soon and potentially more rapidly.

Irrespective of the etiology of the preterm delivery however, the underlying mechanism by which the myometrium contracts remains the same. The central process in the generation of force during a contraction in MSM is the calcium dependent cyclical interaction of the 20 kDa chain of myosin with actin (Word et al. J Clin Invest 92 (1), 29 (1993)). This interaction is fundamentally controlled by the relative equilibrium in activity of myosin light chain kinase and myosin light chain phosphatase, which is in turn profoundly influenced by intracellular calcium concentrations [Ca²⁺]ᵢ (Blanks et al. Best Pract Res Clin Obstet Gynaecol 21 (5), 807 (2007)). Rises in [Ca²⁺]ᵢ in MSM are essential for contraction (Shmygol et al. Ann N Y Acad Sci (2007)). Several groups of investigators working on different species have confirmed that the primary source for the observed rise in [Ca²⁺]ᵢ is extracellular (Parkington et al. J Physiol 514 (Pt 1), 229 (1999); Coleman et al. J Physiol 399, 13 (1988); Luckas et al. Am J Obstet Gynecol 181 (2), 468 (1999)). Furthermore, detailed experiments have confirmed that in intact (not cultured) myometrium extracellular calcium entry is dependent on the opening of voltage gated L-type calcium channels. Voltage gated calcium entry (Parkington et al. J Physiol 514 (Pt 1), 229 (1999); Shimigol et al. J Physiol 511 (Pt 3), 803 (1998); Brown et al. Am J Physiol Cell Physiol 292 (2), C832 (2007)) has been confirmed in a number of species (Luckas et al. Am J Obstet Gynecol 181 (2), 468 (1999); Brown et al. Am J Physiol Cell Physiol 292 (2), C832 (2007); Kupittayanant et al. Bjog 109 (3), 289 (2002)) and is essential for effective contractions.

Within the myometrium, signalling through phosphoinositides is essential in the generation of forceful contractions in response to extracellular stimuli. For example, oxytocin, prostaglandin F_{2α} and prostaglandin E₂ will activate their respective receptors to initiate the hydrolysis of phosphoinositides. Signalling is initiated by conformational change of the cognate receptor leading to coupling to G_{αq/11} G-proteins, which in turn activate phospholipase C (PLC). PLC catalyzes the hydrolysis of phosphatidylinositol-4,5-bisphosphate (PIP₂) to generate inositol-trisphosphate (IP₃) and diacylglycerol (DAG). DAG subsequently activates Protein Kinase C (PKC) which affects multiple cellular processes through phosphorylation, whilst IP₃ binds to receptors on the endoplasmic reticulum (ER) (an intracellular Ca²⁺ store) releasing [Ca²]ᵢ. The released [Ca²⁺]ᵢ then initiates an action potential by activating a calcium dependent cationic conductance.

Considering the importance of both hormonal and ionic mechanisms of uterine contraction there are many potential points at which one can attempt to intervene to establish effective tocolysis (the repression of labour). To add to the difficulty of focusing on a single target, the complexity of signalling during uterine contraction is such that functional redundancy can render targeting a single target ineffective.

### SUMMARY OF THE INVENTION

The present inventors surprisingly identified phospholipase C-like 1 (PLC-L1) as being significantly down-regulated in samples from patients in labour versus patients not in labour (confirmed at the mRNA and protein levels). The present inventors also surprisingly identified that PLC-L1 effectively blocks G_{aq/11} signalling irrespective of agonist, thus preventing release of Ca²⁺ from cell stores and rendering the uterus insensitive to stimulatory hormones. The invention thus provides a method of determining whether or not a patient is in labour. The invention also provides methods for inducing labour in a patient, and methods for preventing labour in a patient. Kits for use in such methods are also provided.

Accordingly, the present invention provides a method of determining whether or not a patient is in labour, said method comprising measuring the amount of phospholipase C-like 1 (PLC-L1) in a sample from the patient and thereby determining whether or not the patient is in labour.

The invention also provides:
a kit for determining whether or not a patient is in labour, comprising a means for taking a sample from the patient and a reagent for measuring the amount of PLC-L1;
an oligonucleotide which specifically hybridises to (a) a part of SEQ ID NOs: 3 and/or 4 or (b) a variant with at least 90% homology to SEQ ID NOs: 3 and/or 4;
an oligonucleotide which comprises 50 or fewer consecutive nucleotides from (a) SEQ ID NOs: 1 and/or 2 or (b) a variant sequence with at least 90% homology to SEQ ID NOs: 1 and/or 2;
a method of inducing labour in a patient, said method comprising administering to the patient an inhibitor of PLC-L1 and thereby inducing labour in the patient;
a method of inducing labour in a patient, said method comprising:
(a) measuring the amount of phospholipase C-like 1 (PLC-L1) in a sample from the patient;

(b) comparing the amount of PLC-L1 in the sample with a control amount of PLC-L1;
(c) if the amount in the sample from the patient is equivalent to or increased compared with the control amount administering an inhibitor of PLC-L1 to the patient and thereby inducing labour in the patient;
a kit for inducing labour in a patient, said kit comprising an inhibitor of PLC-L1 and oxytocin, prostaglandin F2_{α} and/or prostaglandin E₂;
a method of preventing labour in a patient, said method comprising administering to the patient PLC-L1 and thereby preventing labour in the patient;
a method of preventing labour in a patient, said method comprising:
(a) measuring the amount of phospholipase C-like 1 (PLC-L1) in a sample from the patient;
(b) comparing the amount of PLC-L1 in the sample with a control amount of PLC-L1;
(c) if the amount in the sample from the patient is decreased compared with the control amount administering PLC-L1 to the patient and thereby preventing labour in the patient; and
a kit for preventing labour in a patient, said kit comprising PLC-L1 and an inihibtor of oxytocin, prostaglandin F2_{α}, prostaglandin E₂, or Atosiban, Terbutaline, Ritodrine and/or Nifedipine.

### DESCRIPTION OF THE FIGURES

FIG. 1 shows the downregulation of PLC-L1 mRNA transcripts in samples from 5 patients in spontaneous labour and 5 patients not in labour.

FIG. 2 shows immunohistochemistry demonstrating expression of PLC-L1 protein in a sample from a patient in spontaneous labour compared with a sample from a patient not in labour. The bottom right-hand panel shows significantly higher amounts of PLC-L1 present in the sample from the patient not in labour.

FIG. 3 shows Western blots from samples from patients in spontaneous labour (TL) compared with samples from patients not in labour (TNL).

FIG. 4 shows Western blots of PLC-L1 with a control siRNA, PLC-L1 siRNA and with overexpression of PLC-L1. Blots for β-actin are also shown as a control.

FIG. 5 shows calcium signalling in response to oxytocin after overexpression (FIG. 5(b)) and knock down (FIG. 5(c)) of PLC-L1 in primary cultured human myometrium from not in labour patients. FIG. 5(a) shows a control.

FIG. 6 shows calcium signalling in response to PGF₂ₐ after overexpression (FIG. 6(b)) and knock down (FIG. 6(c)) of PLC-L1 in human cultured myometrial cells from not in labour patients. FIG. 6(a)) shows a control.

FIG. 7 shows calcium signalling in response to PGE₂ after overexpression (FIG. 7(b)) and knock down (FIG. 7(c)) of PLC-L1 in human cultured myometrial cells from not in labour patients. FIG. 7(a) shows a control.

### DESCRIPTION OF THE SEQUENCE LISTING

SEQ ID NO: 1 shows the cDNA polynucleotide sequence encoding isoform 1 of human PLC-L1

SEQ ID NO: 2 shows the cDNA polynucleotide sequence encoding isoform 2 of human PLC-L1

SEQ ID NO: 3 shows the mRNA sequence of isoform 1 of human PLC-L1

SEQ ID NO: 4 shows the mRNA sequence of isoform 2 of human PLC-L1

SEQ ID NO: 5 shows the amino acid sequence of isoform 1 of human PLC-L1

SEQ ID NO: 6 shows the amino acid sequence of isoform 2 of human PLC-L1

SEQ ID NOs: 7-10 show sequences used in the Examples

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a nucleic acid sequence” includes two or more such sequences, and the like.

All publications, patents and patent applications cited herein, whether supra or infra, are hereby incorporated by reference in their entirety.

### Method of Determining Whether or not a Patient is in Labour

The present invention relates to a method of determining whether or not a patient is in labour. The patient is typically a human woman, but may also be a female domestic, companion (such as a dog, cat etc) or livestock animal. The patient is pregnant. Typically, the patient is at a late stage in pregnancy. For example, a human woman is typically in the third trimester of pregnancy (at least 28 weeks gestation). The patient is preferably at least 20, 21, 22, 23, 24, 25, 26, 27, 28, 29, 30, 31, 32, 33, 34, 35, 36, 37, 38, 39, 40, or more than 40 weeks (such as 41 or 42 weeks) gestation (or intermediate values thereof). Gestation is the time in which a foetus develops, beginning with fertilization and ending at birth. Labour is the culmination of a gestation period that results in expulsion of the foetus (newborn) from the patient (birth).

The method of the present invention involves measuring the amount of phospholipase-C like 1 (PLC-L1) in a sample from the patient. In humans, two isoforms of the PLC-L1 protein exist. These are created by alternative splicing. The second isoform lacks amino acids 1 to 98 of the first isoform.

SEQ ID NOs: 1 and 2 show the cDNA sequences of both isoforms of human PLC-L1. SEQ ID NOs: 3 and 4 then show the mRNA sequences of the two isoforms. The amino acid sequence of isoform 1 of human PLC-L1 is shown in SEQ ID NO: 5 and the amino acid sequence of isoform 2 is shown in SEQ ID NO: 6. Any sequencing coding for PLC-L1 (RNA or DNA) can be detected and measured in the methods of the present invention. Any of SEQ ID NOs: 1 to 6 can be detected and measured in the method of the present invention. The cDNA, mRNA and amino acid sequences of PLC-L1 from other species are also known and can be accessed via databases such as http://www.uniprot.org and http://www.ncbi.nlm.nih.gov/genbank/.

PLC-L1 is structurally similar to other PLC family members and retains the phosphoinositide-binding plekstrin homology domain, and hence the ability to bind phosphoinositides (Kenematsu et al. Biochem J 313:319-325 (1996)), especially IP₃ (Takeuchi et al. Biochem J 318:561-568 (1996)). One key characteristic that differentiates PLC-L1 within the PLC family is a lack of conservation of residues Glu-390 and His-356 within the catalytic domain critical for PLC activity (Essen et al. Nature 380:595-602(1996); Kenematsu et al. Eur J Biochem 267:2731-2737(2000)). As a consequence of these altered amino acids PLC-L1 lacks catalytic activity (Kenematsu et al. Biochem J 313:319-325 (1996); Kenematsu et al. J Biol. Chem. 267:6518-6525 (1992)). One further characteristic that defines PLC-L1 from other family members is its subcellular location. In contrast to other PLC proteins, PLC-L1 resides near the endoplasmic reticulum (Kenematsu et al. Biochem J 313:319-325 (1996)), the site of action of IP₃-mediated Ca²⁺ mobilization.

The method of the invention comprises measuring the amount of PLC-L1 in a sample from the patient and thereby determining whether or not the patient is in labour. The method of the invention comprises measuring the level of PLC-L1 in a sample from the patient and thereby determining whether or not the patient is in labour. The method of the invention comprises measuring the concentration of PLC-L1 in a sample from the patient and thereby determining whether or not the patient is in labour

The amount of PLC-L1 mRNA may be measured in the sample. Methods for extracting, detecting and measuring amounts of a particular mRNA are known in the art. Any suitable method may be used in the present invention. Methods include microarrays, Northern blotting, nuclease protection assays, in situ hybridization, reverse transcription-polymerase chain reaction (RT-PCR) and or RNA-seq (whole transcriptome sequencing).

The cDNA sequences encoding isoforms 1 and 2 of human PLC-L1 are shown in SEQ ID NOs: 1 and 2, and the mRNA sequences of both isoforms are shown in SEQ ID NOs: 3 and 4. Typically, for a human patient, the amount of mRNA of SEQ ID NO: 3 and/or 4 is measured. However, amounts of variants of SEQ ID NO: 3 and/or 4 may also be measured. These variants reflect the slight differences in PLC-L1 sequence that occur naturally between humans. In particular, variants of SEQ ID NOs: 3 and 4 have a sequence which varies from that of SEQ ID NOs: 3 and 4, but still codes for expression of a functional PLC-L1 protein. PLC-L1 protein is considered to be functional if it binds IP₃, is located at or near the endoplasmic reticulum and lacks catalytic activity. Such variants typically comprise a sequence that is at least 90%, 95%, 97% or 99% homologous based on nucleotide identity to SEQ ID NOs: 3 and 4 over the entire sequence. Methods for determining homology are discussed below.

As these variants only reflect the slight differences in PLC-L1 sequences that occur naturally between humans, the techniques described above can be used to detect and measure such variant mRNAs as well as mRNAs of SEQ ID NOs: 3 and 4. For example, the stringency of hybridisation of probes used in such techniques can be varied so that natural variants of SEQ ID NOs: 3 and 4 will still be detected together with mRNA of SEQ ID NOs: 3 and 4.

As mentioned above, amino acid, mRNA and DNA sequences of PLC-L1 from species other than humans are known and can be accessed via public databases. From these sequences, the skilled person could readily detect and measure PLC-L1 mRNA from such other species, in order to carry out the method of the invention. Methods for mRNA extraction, detection and quantification are discussed above.

Alternatively, amounts of PLC-L1 protein may be measured. Methods for extracting, detecting and measuring amounts of a protein in a sample are well known in the art and any suitable method may be employed in the present invention. Methods may comprise contacting the sample with an antibody capable of binding to PLC-L1. For example immunohistochemistry or Western blotting may be employed, then used to quantify the amount of PLC-L1 present. ELISA could also be used. ELISA is a heterogeneous, solid phase assay that requires the separation of reagents. ELISA is typically carried out using the sandwich technique or the competitive technique. The sandwich technique requires two antibodies. The first specifically binds PLC-L1 and is bound to a solid support. The second antibody is bound to a marker, typically an enzyme conjugate. A substrate for the enzyme is used to quantify the PLC-L1-antibody complex and hence determine the amount of PLC-L1 in a sample. The antigen competitive inhibition assay also typically requires an PLC-L1-specific antibody bound to a support. A PLC-L1-enzyme conjugate is added to the sample (containing PLC-L1) to be assayed. Competitive inhibition between the PLC-L1-enzyme conjugate and unlabeled PLC-L1 allows quantification of the amount of PLC-L1 in a sample. The solid supports for ELISA reactions preferably contain wells.

The present invention may also employ methods of measuring PLC-L1 that do not comprise antibodies. For example, High Performance Liquid Chromatography (HPLC) separation and fluorescence detection may be used as a method of determining the PLC-L1 amount. Alternatively, spectroscopic methods may also be employed.

The amino acid sequences of isoforms 1 and 2 of human PLC-L1 are shown in SEQ ID NOs: 5 and 6. Typically, for a human patient, amounts of protein comprising SEQ ID NOs: 5 and/or 6 are measured in the method of the present invention. However, amounts of variants of SEQ ID NOs: 5 and 6 may also be measured. These variants reflect the slight differences in PLC-L1 sequence that occur naturally between humans. In particular, variants of SEQ ID NOs: 5 and 6 have an amino acid sequence, which varies from that of SEQ ID NOs: 5 and 6, but still forms a functional PLC-L1 protein. PLC-L1 protein is considered to be functional if it binds IP₃, is located at or near the endoplasmic reticulum and lacks catalytic activity. Such variants typically comprise a sequence that is at least 90%, 95%, 97% or 99% homologous based on amino acid identity to SEQ ID NO: 5 or 6 over the entire sequence. Methods for determining homology are discussed below.

Such variants can readily be detected and measured, together with proteins comprising SEQ ID NOs: 5 and 6 themselves, using the techniques and methods for quantitating proteins described above. These methods can often tolerate slight changes in amino acid sequence, such as the changes that occur naturally between humans. For instance, methods may employ antibodies directed against an epitope in SEQ ID NOs: 5 and 6 which is conserved in most, if not all humans. Furthermore, methods could readily be adapted for any commonly occurring variants.

In the method of the invention, the amount of PLC-L1 is typically measured in vitro in a sample from the patient. The sample may be any sample in which the amount of PLC-L1 changes when a patient is in labour, compared with when the patient is not in labour. The sample may comprise a body fluid or a tissue sample from the patient. The sample is preferably a urine, blood, plasma, serum, cervical or uterine sample from the patient. The cervical or uterine sample may be a fluid sample taken from the cervix or uterus, or a tissue sample taken from the cervix or uterus (or both). Methods for obtaining such samples are well known in the art. In particular, cervical samples can be obtained via a cervical swab or scrape. The sample may be processed prior to being assayed. For example, blood samples may be centrifuged. Amounts of PLC-L1 in the sample are typically measured immediately after the sample has been obtained from the patient.

In the method of the invention, the amount of PLC-L1 is measured in a sample from the patient in order to determine whether or not a patient is in labour. A decreased amount of PLC-L1 in the sample is indicative that the patient is in labour. A person skilled in the art is capable of determining whether or not the amount of PLC-L1 is decreased in the patient. Comparisons may be made with a pre-determined amount in the patient itself, or with known control amounts in other patients as discussed in more detail below.

In order to measure whether or not a patient is in labour, the method of the present invention preferably comprises a first step of measuring the amount of PLC-L1 in a sample from the patient. The amount of PLC-L1 in the sample from the patient is then preferably compared with a control amount of PLC-L1 (or a control level of PLC-L1 or a control concentration of PLC-L1). A decreased amount in the sample from the patient compared with the control amount indicates that the patient is in labour. On the other hand, an equivalent or increased (or higher) amount in the sample from the patient compared with the control amount indicates that the patient is not in labour. In other words, no significant difference in the amount in the sample from the patient compared with the control amount indicates that the patient is not in labour.

In some instances, the control amount of PLC-L1 may be derived from measuring the amount of PLC-L1 in samples from comparable pregnant patients. Comparable pregnant patients are patients who are also preferably in the late stages of pregnancy, e.g. the third trimester (at least 28, 29, 30, 31, 32, 33, 34, 35, 36, 37, 38, 39, 40 or more (such as 41 or 42) weeks gestation) for human women. Comparable pregnant patients may, for example, be at a gestation length of at least 20, 21, 22, 23, 24, 25, 26, 27, 28, 29, 30, 31, 32, 33, 34, 35, 36, 37, 38, 39, 40 weeks or more (such as 41 or 42 weeks). However, these patients are not in labour. Standard tests are known for determining whether or not a patient is in labour. These include internal examinations, ultrasound determination of cervical length and detecting foetal fibronectin.

The control amount of PLC-L1 is preferably a range of all the amounts of PLC-L1 present in samples from comparable pregnant patients who are not in labour. The control amount may also refer to the average amount of all the amounts of PLC-L1 present in the samples from the comparable pregnant patients who are not in labour. The sample types obtained from the comparable pregnant patients in which control amounts of PLC-L1 are determined may be any of those described above. The amounts of PLC-L1 in these samples may be determined by any of the methods described above. The control amount may be determined by measuring the amount of PLC-L1 in samples from any appropriate number of comparable pregnant patients who are not in labour. Typically, amounts of PLC-L1 in samples from at least 5, at least 10, at least 20, at least 50 or at least 100 comparable pregnant patients may be measured. Amounts of PLC-L1 in samples from even higher numbers of comparable pregnant patients may also be measured.

Alternatively, the control amount of PLC-L1 may be determined by measuring PLC-L1 in a sample from the patient at an earlier stage in pregnancy. In other words, the control amount may be a previously recorded PLC-L1 measurement(s) from the same patient at an earlier stage in pregnancy. An earlier stage in pregnancy preferably still refers to a late stage in pregnancy. This may be the third trimester in human women (at least 28, 29, 30, 31, 32, 33, 34, 35, 36, 37, 38, 39, 40 or more (such as 41 or 42) weeks gestation). However, an earlier stage in pregnancy may refer to any earlier stage in pregnancy, but is typically a gestation length of at least 20, 21, 22, 23, 24, 25, 26, 27, 28, 29, 30, 31, 32, 33, 34, 35, 36, 37, 38, 39, 40 weeks or more (such as 41 or 42 weeks). At this earlier stage the patient was known not to be in labour. Standard tests which are used to determine whether or not a woman is in labour are mentioned above.

In some cases, only a single control amount of PLC-L1 is determined in a sample from the patient at an earlier stage in pregnancy i.e. the control amount is a single previously recorded measurement from the same patient at an earlier stage in pregnancy (when the patient is not in labour). In some cases, the control amount may be determined by repeatedly measuring the amount of PLC-L1 in different samples from the patient at an earlier stage in pregnancy. In other words, amounts of PLC-L1 are measured in a number of samples from the patient at an earlier stage in pregnancy. In this case, the control amount of PLC-L1 then typically refers to the range of amounts of PLC-L1 measured in these samples. The control amount may also refer to the average amount of PLC-L1 present in the samples from the earlier stages in pregnancy. Again, samples may be any of those described above.

The control amount of PLC-L1 may refer to either PLC-L1 mRNA or PLC-L1 protein, depending on which of these is measured in the sample from the patient who may or may not be in labour. In order to allow an effective comparison, the control amount has the same units as the measured amount of PLC-L1 in the sample from the patient who may or may not be in labour. Furthermore, control values are typically obtained under the same conditions as those under which the method of the invention is carried out. For example, control amounts are typically determined using the same method of PLC-L1 detection and measurement as used in the method of the present invention.

The control amount is obtained separately from the method of the invention. For instance, control amounts may be obtained beforehand and recorded e.g. on a computer.

For measurement of PLC-L1 mRNA, control amounts may be from about 50 to 230 transcripts per million and are typically from about 70 to about 230 transcripts per million when determined using RNA-seq. The control amount may also be from about 50 to 200 transcripts per million, from about 50 to 150 transcripts per million, from about 80 to 200 transcripts per million, from about 100 to about 200 transcripts per million, or from about 125 to about 175 transcripts per million where coverage per transcriptome base is 20-30. An average control amount of PLC-L1 mRNA is preferably about 70, 80, 90, 100, 110, 120, 130, 140, 150, 160 or 170 transcripts per million as determined by RNA-seq where coverage per transcriptome base is 20-30.

The method of the present invention preferably involves determining that a patient is in labour if a decreased amount of PLC-L1 is measured in the sample from the patient compared with the control amount. In other words, a patient is determined to be in labour if the amount of PLC-L1 is lower in the sample from the patient compared with the control amount.

When the control amount is a range of amounts of PLC-L1, a patient may be determined to be in labour based on the spread of the control data, the difference between the control data and the amount of PLC-L1 measured in the sample from the patient, and calculated confidence levels. A person skilled in the art is capable of doing this using standard methods.

A patient may be determined to be in labour if the amount of PLC-L1 in the sample from the patient is decreased compared with the lowest amount of PLC-L1 in the control amount range (either from samples from comparable pregnant patients not in labour or from the patient at an earlier stage in pregnancy).

Preferably, a patient is determined to be in labour if the amount of PLC-L1 in the sample from the patient is decreased by at least 30% (i.e. at least 30% lower), at least 40%, at least 50%, at least 60%, at least 70%, at least 80%, at least 90% or at least 95% compared with the lowest control amount of PLC-L1 in the range of control amounts. Alternatively, the patient may be determined to be in labour if the amount of PLC-L1 in the sample from the patient is decreased compared to the average control amount of PLC-L1 (either from samples from comparable pregnant patients not in labour or from the patient at an earlier stage in pregnancy). Preferably, a patient is determined to be in labour if the amount of PLC-L1 in the sample from the patient is decreased by at least 50%, at least 60%, at least 70%, at least 80%, at least 90% or at least 95% compared with the average control amount of PLC-L1. If only a single measurement of PLC-L1 is taken for the patient at an earlier stage in pregnancy, then the patient is typically determined to be in labour if the amount of PLC-L1 in the sample from the patient is decreased by at least 50%, at least 60%, at least 70%, at least 80%, at least 90% or at least 95% compared with the amount of PLC-L1 at the earlier stage in pregnancy.

For measurement of PLC-L1 mRNA, a patient is typically determined to be in labour if the amount of PLC-L1 mRNA in the sample from the patient is from about 5 to about 40 transcripts per million when the control amount is from about 70 to about 230 transcripts per million, or from about 5 to about 30 transcripts per million when the control amount is from about 50-150 transcripts per million (as determined by RNA-seq where coverage per transcriptome base is 20-30). Alternatively, a patient may be determined to be in labour if the amount of PLC-1 mRNA present in the sample from the patient is less than about 70, less than about 60 or less than about 50 transcripts per million as determined by RNA-seq where coverage per transcriptome base is 20-30. Preferably, the amount of PLC-1 mRNA present in the sample from the patient is less than about 40, less than about 30, less than about 20, less than about 10 or less than about 5 transcripts per million as determined by RNA-seq where coverage per transcriptome base is 20-30.

The method of the present invention may further comprise measuring the amount of oxytocin, prostaglandin F2_{α} and/or prostaglandin E₂ present in a sample from the patient. Oxytocin, prostaglandin F2_{α}, and prostaglandin E₂ are hormones that are released during labour and so may be used as additional indicators of whether or not a patient is in labour. The sample may be the same sample in which the amount of PLC-L1 is measured. Alternatively, the amount of oxytocin, prostaglandin F2_{α} and/or prostaglandin E₂ may be measured in a different sample, provided that the sample is obtained at a similar point in time to the sample in which the amount of PLC-L1 is measured. Samples may be any of those described above.

The amount of oxytocin, prostaglandin F2, and prostaglandin E₂ may be measured by any suitable assay method. For example, immunoassays, ELISA, or spectroscopic methods. Methods for detection and measurement of these hormones are known in the art.

As oxytocin, prostaglandin F2_{α} and prostaglandin E₂ are released during labour, an increase in the amount of these is indicative that the patient is in labour. Therefore in the method of the present invention, a patient is typically determined to be in labour if amounts of PLC-L1 are decreased and amounts of oxytocin, prostaglandin F2_{α} and/or prostaglandin E₂ are increased. As for PLC-L1, an increase in oxytocin, prostaglandin F2_{α} and/or prostaglandin E₂ may be measured relative to control amounts. The control amounts of oxytocin, prostaglandin F2_{α} and/or prostaglandin E₂ may be determined as described above for the control amounts of PLC-L1.

The method of the present invention may be used repeatedly in order to determine whether or not a patient is in labour, or to assess a patient's stage of labour.

### Kit for Determining Whether or not a Patient is in Labour

The present invention also relates to a kit for determining whether or not a patient is in labour. The kit comprises a means for taking a sample from a patient and a reagent for measuring the amount of PLC-L1. The kit thereby allows the determination of whether or not a patient is in labour, depending on the measured amount of PLC-L1.

The means for taking a sample from the patient may be any suitable means depending on the type of sample to be obtained. For example, for a blood, plasma or serum sample a needle may be provided. Alternatively, for a cervical sample a cervical swab or scrape may be provided. The reagent(s) for measuring the amount of PLC-L1 may be any suitable reagent for the detection and/or measurement of amounts of PLC-L1. These reagent(s) may be capable of detecting and/or measuring amounts of PLC-L1 mRNA or PLC-L1 protein. For example, reagents for detecting PLC-L1 protein may include antibodies that specifically bind PLC-L1 (an example of an anti-PLC-L1 antibody is mentioned below). The kit may, for example, comprise a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)₂ or Fv fragment.

The kit may additionally comprise one or more other reagents or instruments which enables the method mentioned above to be carried out. Such reagents include suitable buffers, means to extract/isolate PLC-L1 from the sample or a support comprising wells on which quantitative reactions can be done. The kit may, optionally, comprise instructions to enable the kit to be used in the method of invention or details regarding patients on which the method may be carried out.

Furthermore, the kit may comprise reagent(s) for measuring the amount of oxytocin, prostaglandin F_{2α}, and/or prostaglandin E₂. These reagents may be any suitable reagent for detection of oxytocin, prostaglandin F_{2α}, and/or prostaglandin E₂. In particular, the reagent may be any of those described above for measuring amounts of PLC-L1 (e.g. antibodies).

### Method of Inducing Labour in a Patient

The invention also relates to a method of inducing labour in a patient. The method comprises administering to the patient an inhibitor of PLC-L1 and thereby inducing labour in the patient. Inhibiting PLC-L1 results in a sensitisation of uterine myocytes to uterotonins, and upon stimulation of receptors coupled to Gαq, Ca²⁺ release from stores, which in turn results in the induction of labour. The invention also relates to an inhibitor of PLC-L1 for use in a method of inducing labour in a patient, said method comprising administering the inhibitor of PLC-L1 to the patient and thereby inducing labour. Furthermore, the invention relates to an inhibitor of PLC-L1 for use in the manufacture of a medicament for inducing labour in a patient.

The patient is pregnant. The patient is typically a human woman, but may also be a female domestic, companion (such as a dog, cat etc) or livestock animal. The patient is usually at a late stage in pregnancy. For example, a human woman is typically in the third trimester of pregnancy (at least 28, 29, 30, 31, 32, 33, 34, 35, 36, 37, 38, 39, 40, or more than 40 (such as 41 or 42) weeks gestation). The method of inducing labour may also be used to induce labour in other circumstances, for example where there are health risks to either the foetus or patient.

Before the method of inducing labour is performed, the patient may be determined to not be in labour using the method described above. For example, the method of inducing labour may comprise a first step of measuring the amount of PLC-L1 in a sample from the patient. The amount of PLC-L1 in the sample may then be compared to a control amount of PLC-L1. Both of these steps may be performed as described above. If the amount in the sample from the patient is equivalent or increased compared with the control amount the patient is administered an inhibitor of PLC-L1 and labour is thereby induced in the patient.

An inhibitor of PLC-L1 may be anything that reduces amounts of PLC-L1 present in the patient, or that prevents the functionality of PLC-L1. The PLC-L1 being inhibited typically comprises the amino acid sequence of SEQ ID NO: 5 and/or 6 (both isoforms of human PLC-L1) or a naturally occurring variant thereof. Such variants of PLC-L1 are described above. In other animals, the amino acid sequence of PLC-L1 may be identified from databases such as http://www.uniprot.org. Again, the PLC-L1 may be a naturally occurring variant as described above.

PLC-L1 is considered to be functional if it binds IP₃, is located at or near the endoplasmic reticulum and lacks catalytic activity. Inhibitors of PLC-L1 may block this functionality, for example by preventing the protein from localising at or near the endoplasmic reticulum. Inhibitors may also prevent PLC-L1 from binding and sequestering IP₃. Suitable inhibitors include small molecules and antibodies that bind to PLC-L1. An example of an anti-PLC-L1 antibody is Sigma HPA031849.

Inhibitors of PLC-L1 may also reduce amounts of PLC-L1 present in the patient, for example by knocking down expression of PLC-L1. Antisense and siRNA technology for knocking down protein expression are well known in the art and standard methods can be employed to knock down expression of PLC-L1. Both antisense and siRNA technology interfere with mRNA. Antisense oligonucleotides interfere with mRNA by binding to (hybridising with) a section of the mRNA. The antisense oligonucleotide is therefore designed to be complementary to the mRNA (although the oligonucleotide does not have to be 100% complementary as discussed below). In other words, the antisense oligonucleotide may be a section of the cDNA. Again, the olignucleotide sequence may not be 100% identical to the cDNA sequence. This is also discussed below. siRNA involves double-stranded RNA (double-stranded versions of these oligonucleotides). siRNAs for knocking down PLC-L1 are shown in the Examples.

Accordingly, the present invention provides an oligonucleotide which specifically hybridises to a part of (a) SEQ ID NOs: 3 and/or 4 (PLC-L1 mRNA) or (b) a variant sequence with at least 90% homology to SEQ ID NOs: 3 and/or 4 as defined above. Oligonucleotides are short nucleotide polymers which typically have 50 or fewer nucleotides, such 40 or fewer, 30 or fewer, 22 or fewer, 21 or fewer, 20 or fewer, 10 or fewer or 5 or fewer nucleotides. The oligonucleotide of the invention is preferably 20 to 25 nucleotides in length, more preferably 21 or 22 nucleotides in length.

The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the oligonucleotide can be oxidized or methylated. One or more nucleotides in the oligonucleotide may be damaged. For instance, the oligonucleotide may comprise a pyrimidine dimer. Such dimers are typically associated with damage by ultraviolet light.

The nucleotides in the oligonucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The invention also provides an oligonucleotide which comprises 50 or fewer consecutive nucleotides from (a) SEQ ID NOs: 1 and/or 2 (cDNA sequences of human PLC-L1) or (b) a variant sequence with at least 90% homology to SEQ ID NOs: 1 and/or 2 as defined above. The oligonucleotide may be any of the lengths discussed above. It is preferably 21 or 22 nucleotides in length. The oligonucleotide may comprise any of the nucleotides discussed above, including the modified nucleotides.

The oligonucleotide can be nucleic acids, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The oligonucleotide may comprise any of the nucleotides discussed above, including the modified nucleotides. The oligonucleotide is preferably RNA.

The oligonucleotide is preferably single stranded. The oligonucleotide may be double stranded i.e. siRNA comprising an oligonucleotide with 50 or fewer consecutive nucleotides from SEQ ID NOs: 3 and/or 4 and 50 or fewer consecutive nucleotides from SEQ ID NOs: 1 and/or 2. siRNA technology is well known in the art and standard methods can be employed to knock down PLC-L1 in the present invention.

An oligonucleotide of the invention preferably specifically hybridises to a part of a SEQ ID NO: 3 and/or 4, hereafter called the target sequence. The length of the target sequence typically corresponds to the length of the oligonucleotide. For instance, a 21 or 22 nucleotide oligonucleotide typically specifically hybridises to a 21 or 22 nucleotide target sequence. The target sequence may therefore be any of the lengths discussed above with reference to the length of the oligonucleotide. The target sequence is typically consecutive nucleotides within the polynucleotide of the invention.

An oligonucleotide “specifically hybridises” to a target sequence when it hybridises with preferential or high affinity to the target sequence but does not substantially hybridise, does not hybridise or hybridises with only low affinity to other sequences.

An oligonucleotide “specifically hybridises” if it hybridises to the target sequence with a melting temperature (Tₘ) that is at least 2° C., such as at least 3° C., at least 4° C., at least 5° C., at least 6° C., at least 7° C., at least 8° C., at least 9° C. or at least 10° C., greater than its Tₘ for other sequences. More preferably, the oligonucleotide hybridises to the target sequence with a Tₘ that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tₘ for other nucleic acids. Preferably, the portion hybridises to the target sequence with a Tₘ that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tₘ for a sequence which differs from the target sequence by one or more nucleotides, such as by 1, 2, 3, 4 or 5 or more nucleotides. The portion typically hybridises to the target sequence with a Tₘ of at least 90° C., such as at least 92° C. or at least 95° C. Tₘ can be measured experimentally using known techniques, including the use of DNA microarrays, or can be calculated using publicly available Tₘ calculators, such as those available over the internet.

Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). Hybridisation can be carried out under low stringency conditions, for example in the presence of a buffered solution of 30 to 35% formamide, 1 M NaCl and 1% SDS (sodium dodecyl sulfate) at 37° C. followed by a 20 wash in from 1×(0.1650 M Na⁺) to 2×(0.33 M Na⁺) SSC (standard sodium citrate) at 50° C. Hybridisation can be carried out under moderate stringency conditions, for example in the presence of a buffer solution of 40 to 45% formamide, 1 M NaCl, and 1% SDS at 37° C., followed by a wash in from 0.5×(0.0825 M Na⁺) to 1×(0.1650 M Na⁺) SSC at 55° C. Hybridisation can be carried out under high stringency conditions, for example in the presence of a buffered solution of 50% formamide, 1 M NaCl, 1% SDS at 37° C., followed by a wash in 0.1×(0.0165 M Na⁺) SSC at 60° C.

The oligonucleotide of the invention may comprise a sequence which is substantially complementary to the target sequence. Typically, the oligonucleotides are 100% complementary. However, lower levels of complementarity may also be acceptable, such as 95%, 90%, 85% and even 80%, Complementarity below 100% is acceptable as long as the oligonucleotides specifically hybridise to the target sequence. An oligonucleotide may therefore have 1, 2, 3, 4, 5, 6, 7, 8, 9, 10 or more mismatches across a region of 5, 10, 15, 20, 21, 22, 30, 40 or 50 nucleotides.

Oligonucleotides may be synthesised using standard techniques known in the art. Alternatively, oligonucleotides may be purchased.

In the method of the invention, the inhibitor of PLC-L1 is administered to the patient in order to induce labour. Inhibitors of PLC-L1 may be administered to the patient in any appropriate way. In the invention, inhibitors may be administered in a variety of dosage forms. Thus, they can be administered orally, for example as tablets, troches, lozenges, aqueous or oily suspensions, dispersible powders or granules. They may also be administered parenterally, either subcutaneously, intravenously, intramuscularly, intrasternally, transdermally or by infusion techniques. Inhibitors are typically administered intravaginally. They may also be administered as suppositories. A physician will be able to determine the required route of administration for each particular patient.

The formulation of an inhibitor for inducing labour in accordance with the invention will depend upon factors such as the nature of the exact inhibitor, etc. An inhibitor may be formulated for simultaneous, separate or sequential use.

An inhibitor according to the invention is typically formulated for administration in the present invention with a pharmaceutically acceptable carrier or diluent. The pharmaceutical carrier or diluent may be, for example, an isotonic solution. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

Inhibitors of the invention are typically administered directly to the vagina, cervix or uterus of the patient.

The oligonucleotides maybe naked nucleotide sequences or be in combination with cationic lipids, polymers or targeting systems. The oligonucleotides may be delivered by any available technique. For example, the oligonucleotide may be introduced by needle injection, preferably intradermally, subcutaneously or intramuscularly. Alternatively, the oligonucleotide may be delivered directly across the skin using a oligonucleotide delivery device such as particle-mediated gene delivery. The oligonucleotide may be administered topically to the skin, or to mucosal surfaces for example by intranasal, oral, or intrarectal administration. In the present invention, the oligonucleotides are preferably administered intravaginally.

Uptake of oligonucleotide constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the oligonucleotide to be administered can be altered.

A therapeutically effective amount of an inhibitor is administered to the patient. The dose may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated and the frequency and route of administration. The dose may be provided as a single dose or may be provided as multiple doses, for example taken at regular intervals, for example 2, 3 or 4 doses administered hourly. Preferably, dosage levels of inhibitors are from 5 mg to 2 g.

Typically oligonucleotide inhibitors are administered in the range of 1 pg to 1 mg, preferably to 1 pg to 10 μg nucleic acid for particle mediated delivery and 10 μg to 1 mg for other routes.

The method of inducing labour in a patient according to the invention may also comprise administering to the patient oxytocin, prostaglandin F2_{α} and/or prostaglandin E₂. Doses and administration routes of oxytocin, prostaglandin F2_{α} and prostaglandin E₂ can be determined by a physician as described above. The oxytocin, prostaglandin F2_{α} and prostaglandin E₂ may be administered together with the PLC-L1 inhibitor or separately (either simultaneously in separate doses or at separate time points). The oxytocin, prostaglandin F2_{α} and/or prostaglandin E2 may be co-administered with the inhibitor of PLC-L1. The administration of an inhibitor of PLC-L1 may also be combined with other techniques for inducing labour in a patient. A detailed discussion of such techniques can be found at http://www.nice.org.uk/Guidance/CG70/NiceGuidance/pdf/English.

### Kit for Inducing Labour in a Patient

The present invention also provides a kit for inducing labour in a patient. The kit comprises an inhibitor of PLC-L1 and oxytocin, F2_{α} and/or prostaglandin E₂. The inhibitor of PLC-L1, and the oxytocin, F2_{α} and/or prostaglandin E₂, may be formulated appropriately for administration to the patient as described above. The kit may comprise the inhibitor of PLC-L1, and the oxytocin, F2_{α} and/or prostaglandin E₂, in a single container or in separate containers. The kit may additionally comprise one or more other reagents or instruments which enables the method mentioned above to be carried out.

The kit may, optionally, comprise instructions to enable the kit to be used in the method of invention or details regarding patients on which the method may be carried out.

### Method of Preventing Labour in a Patient

The invention also provides a method of preventing labour in a patient. The method comprises administering to the patient PLC-L1 and thereby preventing labour.

The patient is pregnant. The patient may be any patient as described above. The patient is at a stage in pregnancy where labour is not desirable. For example, the patient may be at a stage in pregnancy where the onset of labour would have significant health risks for the patient or for the foetus.

The patient is typically at risk of preterm labour. The method may be used to prevent preterm labour in patients before 23 weeks gestation. The method may also be used to prevent labour in a patient up to the full gestation term e.g. up until 40 weeks gestation for a human. The patient may also be in labour, or heading towards labour, and the method is used to prevent labour from developing, or from developing further

The method described above may first be used to determine that the patient is in labour. For example, the method of the invention may comprise measuring the amount of PLC-L1 in a sample from the patient. The amount of PLC-L1 in the sample is then compared with a control amount of PLC-L1. Methods of determining amounts of PLC-L1 and control amounts of PLC-L1 are described above. If the amount in the sample from the patient is decreased compared with the control amount PLC-L1 is administered to the patient, thereby preventing labour in the patient. This method is typically used at the onset of labour in order to prevent labour from developing. The method may also be used in situations where there are signs that a patient is about to enter labour and there is a reasonable presumption that labour would follow. However, delivery would not be recommended at that point.

The amino acid sequences of both isoforms of human PLC-L1 are shown in SEQ ID NOs: 5 and 6. The method of the present invention typically comprises administering a protein (polypeptide) of SEQ ID NO: 5 or 6 to the patient in order to prevent labour. However, in other animals PLC-L1 with the appropriate amino acid sequence may be administered.

The protein may also be a variant of SEQ ID NOs: 5 and 6. A variant is a protein that has an amino acid sequence which varies from that of SEQ ID NOs: 5 and 6, and which retains its ability to bind IP₃, localisation at or near the endoplasmic reticulum and lack of catalytic activity. The ability of a variant to bind IP₃ can be assayed using any method known in the art. Furthermore, standard techniques exist for determining the localisation of proteins within the cell and for determining catalytic activity. Techniques include competitive binding assays and fluorescence microscopy.

The variant may be a naturally occurring variant which is expressed naturally, for instance in humans. Alternatively, the variant may be expressed in vitro or recombinantly by a bacterium such as Escherichia coli. Variants also include non-naturally occurring variants produced by recombinant technology.

Over the entire length of the amino acid sequence of SEQ ID NOs: 5 or 6, a variant will preferably be at least 80% homologous to that sequence based on amino acid identity. More preferably, the variant may be at least 85%, at least 90% and more preferably at least 95%, 97% or 99% homologous based on amino acid identity to the amino acid sequence of SEQ ID NOs: 5 or 6 over the entire sequence. There may be at least 80%, for example at least 85%, 90% or 95%, amino acid identity over a stretch of 800 or more, for example 850, 900, 950 or 1000 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

Amino acid substitutions may be made to the amino acid sequences of SEQ ID NOs: 5 and 6 in addition to those discussed above, for example up to 1, 2, 3, 4, 5, 10, 20, 30, 50, 100 or 200 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, | Met | hydrophobic, neutral |
| | neutral | | |
| Cys | polar, hydrophobic, | Asn | polar, hydrophilic, |
| | neutral | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NOs: 5 or 6 may additionally be deleted from the polypeptides described above. Up to 1, 2, 3, 4, 5, 10, 20, 30 or 50 residues may be deleted, or more.

Variants may include fragments of SEQ ID NOs: 5 and 6. Such fragments retain functionality. Fragments may be at least 600, 700, 800 or 900 amino acids in length. One or more amino acids may be alternatively or additionally added to the polypeptides described above.

Alternatively, a polynucleotide encoding PLC-L1 may be administered to the patient in order to prevent labour. A polynucleotide, such as a nucleic acid, is a polymer comprising two or more nucleotides. The nucleotides can be naturally occurring or artificial. Nucleotides can be any of those described above.

The PLC-L1 may be any of those described above. The polynucleotide may have any sequence as long as it codes for functional PLC-L1. Functional PLC-L1 is described above. The polynucleotide sequence preferably comprises SEQ ID NOs: 1 or 2 (cDNA of human PLC-L1) or a variant sequence with at least 80%, 90% or 95% homology based on nucleotide identity to the sequence of SEQ ID NO: 1 or 2 over the entire sequence. There may be at least 80%, for example at least 85%, 90% or 95% nucleotide identity over a stretch of 2,000 or more, for example 2,500, 2,750, or 3,000 or more, contiguous nucleotides (“hard homology”). Homology may be calculated as described above. The polynucleotide sequence may comprise a sequence that differs from SEQ ID NOs: 1 or 2 on the basis of the degeneracy of the genetic code.

Polynucleotide sequences may be derived and replicated using standard methods in the art, for example using PCR involving specific primers. It is straightforward to generate polynucleotide sequences using such standard techniques.

The amplified sequences may be incorporated into a recombinant replicable vector such as a cloning vector. The vector may be used to replicate the polynucleotide in a compatible host cell. Thus polynucleotide sequences encoding the PLC-L1 may be made by introducing a polynucleotide encoding the PLC-L1 into a replicable vector, introducing the vector into a compatible host cell, and growing the host cell under conditions which bring about replication of the vector. The vector may be recovered from the host cell. Suitable host cells for cloning of polynucleotides are known in the art and described in more detail below. The polynucleotide sequence may be cloned into any suitable expression vector. In an expression vector, the polynucleotide sequence encoding a construct is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence. The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used. The host cell typically expresses the construct at a high level. Host cells transformed with a polynucleotide sequence encoding a construct will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter.

The PLC-L1 protein, or the polynucleotide encoding PLC-L1, may be administered to the patient using standard techniques known in the art. The protein or polynucleotide is preferably administered in a therapeutically effective amount. Therapeutically effective amounts are discussed above.

The PLC-L1 protein, or the polynucleotide encoding PLC-L1 may be administered to the patient by any suitable means. In particular, the PLC-L1 protein, or the polynucleotide encoding PLC-L1, may be formulated for administration as described above. Dosage regimes may also be determined as described above. The PLC-L1 is typically administered directly to the cervix or uterus of the patient i.e. intravaginally.

The method of preventing labour in the patient may also comprise administering to the patient an inhibitor of oxytocin, prostaglandin F_{2α} and/or prostaglandin E₂. Inhibitors of oxytocin, prostaglandin F_{2α} and/or prostaglandin E₂ can by any substance which reduces amounts of oxytocin, prostaglandin F_{2α} and/or prostaglandin E₂ present, or that blocks signalling by these hormones. Inhibitors may be small molecules, antibodies, antisense RNA or siRNA. Such inhibitors are described above. Inhibitors could also interfere with oxytocin, prostaglandin F_{2α} and/or prostaglandin E₂ signalling e.g. through preventing binding of these hormones to their receptors. Again, such inhibitors could be small molecules or antibodies.

The PLC-L1 could also be administered in combination with other tocolytics known in the art for preventing labour. Known tocolytics include Terbutaline, Ritodrine and Nifedipine, Atosiban. These tocolytics, together with the PLC-L1, can be formulated appropriately for administration as described above.

### Kit for Preventing Labour in a Patient

The invention also relates to a kit for preventing labour in a patient. The kit comprises PLC-L1 and additional inhibitor(s) of labour. The PLC-L1 can be PLC-L1 protein, or a polynucleotide encoding PLC-L1, as described above. Furthermore, the inhibitors of labour may be any of those described above. The PLC-L1 and the additional inhibitor(s) of labour may be formulated appropriately for administration to the patient as described above. The kit may comprise the PLC-L1 and the inhibitor(s) in a single container or in separate containers. The kit may additionally comprise one or more other reagents or instruments which enables the method mentioned above to be carried out.

The kit may, optionally, comprise instructions to enable the kit to be used in the method of invention or details regarding patients on which the method may be carried out.

The following Examples illustrate the invention.

### Examples

### Materials and Methods—1

Each tissue was placed in sterile foil and broken up with a mallet on the bench. For each sample about 0.3 g of myometrium was transferred to a sheet of sterile foil and then pulverised by repeatedly placing the foil-wrapped samples into liquid nitrogen, which were then smashed with a mallet. Each sample was transferred to a 50 ml falcon tube, 1 ml of RNA-Stat60 reagent was added and the tube was stored on dry ice. Each sample was thawed, homogenised with a LabGen® 7 Series Homogenizer (220 VAC) and then stored at room temperature for 5 min before they were transferred to 1.5 ml eppendorf tubes. 0.2 ml of chloroform was added and each tube was vortexed for 15 s. The samples were then put back on dry ice for 20 min, thawed and then centrifuged for 30 min. The aqueous layer was removed into new tubes and then 0.5 ml of ice cold isopropanol was added, discarding the phenol chloroform layer. The tubes were then gently inverted. Samples were stored at room temperature for 10 min and then centrifuged for 15 min.

The supernatant was removed and the pellet was washed in 1 ml 75% ethanol and centrifuged for 15 min. The supernatant was removed and the pellet was air-dried for 20 min. The pellet was then dissolved in 87.5 μl of nuclease free water. DNase treatment and RNA purification was then performed according to Appendix E (DNase Digestion of RNA before RNA cleanup) of the Qiagen RNeasy Mini handbook using the Qiagen RNase-free DNase set and the Qiagen RNeasy. The RNA was eluted in afinal volume of 60 μl of nuclease-free water and stored at −80° C.

All centrifugation was performed at 13,000 g at 4° C. The homogenizer was cleaned thoroughly between tissue samples with 100% ethanol washes.

RNA quality was assessed with an Agilent Bioanalyser with a cut-off RIN score of 6. RNA-Seq amplification was performed using the NuGEN Ovation RNA-Seq System and cDNA was assessed on a Qubit® Fluorometer. Sequencing was performed on an Illumina GAIIx using 70 bp paired ends reads.

### Materials and Methods—2

Materials:

DMEM+Glutamax-II culture media and fetal bovine serum (FBS), penicillin/streptomycin, PBS and trypsin and Recovery™ cell culture freezing media, fluo-4-AM and collagenase ((type iv) 230 units/mg prepared from Clostridium Histolyticum) were all supplied by Invitrogen (Paisley, UK). All cell culture plastic ware was supplied by Nunc with the exception of 35 mm glass-bottomed collagen-coated cell culture dishes which were obtained from MatTek (Ashland, Mass., U.S.A.). Human PLC-L1 cDNA glycerol stocks, SmartPool siGenome Human PLC-L1 siRNA (GUAGGGAGCUCUCUGAUUU (SEQ ID NO: 7), GAAGAAAGUUCGGGAAUAU (SEQ ID NO: 8), GGUAAUGGCUCAACAGAUG (SEQ ID NO: 9) and GCACAGAAGCGCAGUCUUU (SEQ ID NO: 10), siGenome non-targetting siRNA pool and siRNA buffer were all obtained from Thermo Fisher Scientific (Hemel Hempstead, UK) with transfection achieved via Amaxa™ Basic Nucleofector™ Kits supplied by Lonza (Basel, Switzerland). LB broth and kanamycin were supplied by Sigma (Poole, UK) and GeneJet™ maxiprep kits by Fisher Scientific (Loughborough, UK). For IHC/ICC, Polysine® glass slides were supplied by Thermo Scientific (Hemel Hempstead, UK) and BD Falcon tissue-culture-treated 4-chamber culture slides were supplied by BD Biosciences (Bedford, Miss., U.S.A). OCT cryo-embedding matrix and ProLong Gold antifade reagent with DAPI were from Fisher Scientific (Loughborough, UK), disposable Feather R35 microtome blades were obtained VWR International Ltd (Harlow, UK), Novolink polymer detection kits from Leica (Wetzlar, Germany), 10% neutral buffered formalin from Genta Medical (York, UK) and DPX mountant from Surgipath (Peterborough, U.K). Anti-human PLC-L1 antibody (0.3 mg/ml) was supplied by Sigma (Poole, UK) and Alexa-Fluor® 488 goat anti-rabbit secondary antibody IgG (H+L) (2 mg/ml) by Invitrogen Molecular Probes (Paisley, UK). For western blotting experiments, 10× concentration RIPA buffer was supplied by Millipore (Watford, UK) and contained cOmplete mini protease inhibitor cocktail tablets from Roche (Burgess Hill, UK). NuPage LDS 4× samples buffer, Novex Tris-Glycine SDS running buffer and 20×NuPage Transfer buffer were all obtained from Invitrogen (Paisley, UK). Mini-PROTEAN™ western blotting tanks and 10% TGX gels were supplied by Bio-Rad (Hemel Hempstead, UK), with nitrocellulose and ECL reagents by GE Healthcare (Amersham, UK). Goat anti-rabbit-HRP and rabbit anti-mouse-HRP secondary antibodies were obtained from DAKO (Ely, UK) and β-actin antibody from Sigma (Poole, UK). Oxytocin was supplied by Tocris (Bristol, UK), and prostaglandin E2 and (PGE2) and prostaglandin F2α (PGF2α) from Sigma (Poole, UK). All other chemicals and reagents were supplied by either Sigma (Poole, UK) or Fisher Scientific (Loughborough, UK).

Tissue Collection and Primary Myometrial Cell Culture:

Tissue collection: Whole myometrial biopsies were collected from women undergoing elective and emergency Caesarean section (>36 weeks gestation) to represent non-labouring and labouring myometrial samples, respectively. Patients gave written informed consent with full ethical approval (REC-05/Q2802/107). Biopsies were taken prior to administration of synthetic oxytocin from the upper edge of the incision in the lower uterine segment. To distinguish labouring and non-labouring samples, spontaneous labour was defined as regular contractions (<3 mins apart), membrane rupture and cervical dilation (<2 cm) with no augmentation. For experiments involving cell dissociation and culture, myometrial biopsies (collected from non-labouring women only) were stored in sterile modified Krebs'-Henseleit buffer (composition: NaCl 133 mM, KCl 4.7, Glucose 11.1, MgSO4 1.2, KH2PO4 1.2, CaCl 2.5, TES 10, pH 7.4, 37° C.) at 4° C. for up to 2 hours before use. Where experiments involved frozen tissue sections, biopsies were snap frozen in liquid nitrogen (LN2) immediately on collection, and transferred to −80° C. for storage.

Cell Dissociation:

Whole tissue biopsies from non-labouring women were washed in sterile modified Krebs′-Henseleit buffer to remove superficial blood and were dissected to remove any excess fatty or connective tissues. Biopsies were then diced into small 0.5-1 mm pieces and incubated with 2 mg/ml collagenase (type iv) in DMEM+Glutamax-II media containing penicillin (100 IU/ml) and streptomycin (100 μg/ml)) for 4 hours (or until majority of tissue pieces had been digested) at 37° C. with vigorous agitation. Cells were then collected via centrifugation (1000 rpm, 5 min) and washed with 3 repeated cycles of trituration in PBS (37° C.) and pelleting (1000 rpm, 5 min). Following the final wash, pellets were re-suspended in culture media and plated into 175 cm² flasks as described below. Cell culture: Primary myometrial cells were routinely maintained in 175 cm² flasks in DMEM+Glutamax-II media supplemented with 10% FBS, penicillin (100 IU/ml) and streptomycin (100 μg/ml) at 37° C. in a 95%/5% air/CO2-humidified environment. Cells were sub-cultured at 80% confluency with 0.05% trypsin and gentle agitation. Trypsin within lifted cell suspensions was neutralized by addition of an equal volume of cell growth media (with supplements) and cells were pelleted by centrifugation (1000 rpm, 5 min). Cells were passaged approximately every 3-4 days at 1:10 dilution and never used beyond passage 4. For assessment of cell density, cells within a small aliquot of suspension were counted using a neubauer approved haemocytometer. For long-term storage, cells were grown in 175 cm² flasks until 60-70% confluent and harvested as above. Pelleted cells were re-suspended in Recovery™ cell culture freezing media (3 ml/175 cm² flask), transferred to air-sealed sterile cryovials and frozen at a rate of 1° C./min to −80° C., before transfer to LN₂.

Transfection of Primary Myometrial Cells Cultures:

Purification of cDNA: Ready-transformed bacteria containing the cDNA for human PLC-L1 in a pCR4-TOPO plasmid were purchased as glycerol stocks from Thermo Scientific (Hemel Hempstead, UK). Glycerol stocks were incubated overnight at 37° C. (with vigorous shaking) in LB broth containing 100 μg/ml kanamycin (nb: untransformed cells did not grow in the presence of kanamycin). DNA was purified from bacterial growths via the GeneJet™ maxiprep kit exactly as per the manufacturer's instructions with final DNA concentration in H₂O determined by nano-drop spectrophotometry. For siRNA, purchased stocks were centrifuged and diluted in sterile 1×siRNA buffer to give the desired concentration.

Transfection: On day of transfection, primary myometrial cell cultures were harvested with trypsin, pelleted via centrifugation and re-suspended in DMEM media (with supplements). Cell density was assessed via a neubauer approved haemocytometer and adjusted to 1×10⁶ cells per tube which were subsequently pelleted by centrifugation (1000 rpm, 5 min). All media was carefully removed from the pellet before transfection via an Amaxa™ basic Nucleofector™ kit as per manufacturer's instructions. Briefly, 1×10⁶ cells were re-suspended in 100 μl supplemented Nucleofector™ solution containing either 10 nM (1 pmol/sample) siGenome non-targeting siRNA, 50 nM (5 pmol/sample) SmartPool: siGenome Human PLC-L1 siRNA or 1 m PLC-L1 plasmid DNA. Electroporation proceeded on Nucleofector™ program A-33. Pre-warmed, supplemented DMEM was added to cells following electroporation which were then transferred to appropriate culture dishes for 2 days. Media was changed 18-24 hours after transfection.

Immuno-Histochemistry:

Non-labouring and labouring myometrial biopsies were immediately frozen in LN₂ on collection. Biopsies were embedded in cryomatrix before 5 thick frozen sections were sliced at −20° C. in a CM1850 Cryostat (Leica, Wetzlar, Germany) tissue slicer, and then mounted onto Polysine® glass slides. Sections were immediately fixed in 10% neutral buffered formalin for 5 mins and stained using a Novolink polymer detection kit exactly as per manufacturer's instructions (Leica), with washing achieved with PBS-T (PBS with 0.05% tween-20). Tissue slices were exposed to a 1:500 dilution of primary anti-body targeting human PLC-L1 and incubated overnight at 4° C. Parallel slices were stained with H&E (1 min haematoxilin, 10 s Eosin) to visualize tissue morphology. Slides were dehydrated through graded alcohol (80%, 90% and 100%) and cleared with xylene before fixation in DPX mountant under glass coverslips. Slides were allowed to dry and sealed with nail varnish. Stained slices were imaged using light microscopy (Leica DM200) with background, colour and contrast settings consistent throughout to permit the comparison of different images.

Western Blotting:

Tissue preparation: Non-labouring and labouring myometrial biopsies were snap frozen in LN₂ on collection and mechanically ground on dry-ice into a fine powder using pestle and mortar. Ground biopsies were suspended in ice-cold 1×RIPA buffer (with complete protease inhibitor cocktail tablets (1 tablet/10 ml)) and homogenized for 30-60 s using a LabGen7 7 mm handheld homogenizer (Coleman, Ill., U.S.A). Homogenates were subject to a pre-clearance step (1000 rpm, 5 min, 4° C.) to remove any tissues debris before supernatants across all samples were normalised to 1 mg/ml protein content as determined by the Bradford method (Bradford et al., 1976). Samples were mixed with 100 nM DTT and 25% (v:v) NuPage LDS 4× sample buffer before heating (5 min, 100° C.) and separation via western blotting.

Cell preparation: Cells were grown in 6-well plates and lysed with 150 μl/well RIPA buffer 2 days after transfection. Lysates were normalized for protein and samples prepared exactly as for tissues described above.

Western blotting: Samples (25 μg protein) were loaded into 10% TGX pre-cast acrylamide gels and separated by standard SDS-PAGE electrophoresis techniques and nitrocellulose transfer using the Bio-Rad mini-PROTEAN system. Nitrocellulose blots were blocked in 5% (w:v) powdered milk in PBS-T before detection of PLC-L1 by incubation with an anti-PLC-L1 antibody (1:500 in 5% milk PBS-T, overnight, 4° C.). Blots were washed clear of unbound antibody in PBS-T before addition of anti-rabbit-HRP conjugated secondary antibody (1:1000 in PBS-T, 1 hr, RT). Following further washing in PBS-T, immune-reactive bands were visualized with ECL reagent and standard auto-radiography techniques. To ensure accurate loading of protein into gels, parallel samples were detected for fl-actin (1:100000 in 5% Milk PBS-T, overnight, 4° C.) and anti-mouse-HRP conjugated secondary antibody (1:1000 in PBS-T, 1 hr, RT).

Immuno-Cytochemistry:

Transfection and culture of primary myometrial cells: Where experiments required over-expression and knockdown of PLC-L1, cells were transfected with siRNA targeting PLC-L1, scrambled non-targeting siRNA or DNA encoding PLC-L1 using Amaxa™ Nucleofection™. Cells were seeded into BD Falcon 4-chamber cell-culture treated glass slides and cultured for 1-2 days until 60-70% confluent.

Agonist-mediated stimulation: For experiments requiring agonist stimulation, untransfected cells were seeded into 4-chamber culture slides and cultured for 1-2 days. On day of assay, cells were washed in modified Krebs′-Heinseleit buffer and incubated at 37° C. for 15 minutes. Cells were challenged by 100 nM oxytocin for desired time period with termination by rapid aspiration of buffer, addition of ice-cold 100% methanol and incubation at −20° C. for 10 minutes. Blocking and subsequent steps proceeded as described below.

Immuno-cytochemistry: Cells were washed in PBS and fixed in 100% methanol (−20° C., 10 min). Cells were washed again in PBS before blocking of non-specific sites with incubation in 10% (v:v) goat serum/PBS for 30 min, RT. Cells were then incubated overnight at 4° C. with primary anti-PLC-L1 antibody (1:500 in 1% goat serum/PBS). Cells were washed in 1% goat serum in PBS before incubation with Alex-Fluor® 488 goat anti-rabbit secondary antibody (1:1000 in 1% goat serum/PBS) for 1 hr at RT, with protection from light. Secondary antibody was removed by washing as described above before mounting coverslips with Prolong® Gold anti-fade reagent with DAPI. This was allowed to dry before coverslips were sealed and stored at 4° C. until imaged.

Confocal imaging: Slides were imaged on the stage of a Zeiss Axiovert 200M inverted microscope and visualized with a 40× objective lens. Fluorescence was detected using a Zeiss LSM 510 confocal imaging system (Baltimore, Md., U.S.A.) whereby cells were excited with a krypton/argon laser at 488 nm and emitted light collected above 510 nm for detection of Alexa-Fluor 488. Simultaneously, cells were excited at 350 nm with emitted light collected at 470 nm for the detection of DAPI nuclear staining. To permit a comparison of the fluorescent intensity of various images, exact confocal setting, laser intensities, images brightness and contrast were kept constant throughout.

Confocal Ca²⁺ Imaging:

Cell culture and transfection: Primary cultures of myometrial cells were transfected with either 10 nM non-targeting siRNA, 50 nM SmartPool: Human PLC-L1 siRNA or 1 μg PLC-L1 plasmid DNA using Amaxa nucleofection techniques and cultured in 35 mm glass-bottomed culture dishes for 2 days.

Ca²⁺ imaging: Cells were washed in modified Kreb's-Heinselet buffer and loaded with 5 μM Fluo-4-AM for 1 hr at RT with limited exposure to light. Extracellular fluo-4 was then removed and cells were incubated in 2 ml Kreb's-Heinselet buffer on the stage of a Zeiss Axiovert 200M inverted microscope and visualized with a 40× objective lens. Temperatures were maintained at 37° C. with a peltier unit. Using a Zeiss LSM 510 confocal imaging system (Baltimore, Md., U.S.A.) cells were excited with a krypton/argon laser at 488 nm and emitted light collected above 510 nm. Cells were imaged for 10 minutes to allow any laser-induced Ca²⁺ signaling to subside. For live experiments, cells were challenged with 10 nM oxytocin, 250 nM PGF2α or 2.2 μM PGE2 by 100× concentration agonist bath addition and fluorescence captured by cooled CCD camera at a rate of approximately one frame per second. Videos were visualized with LSM work station image analysis software. Data analysis: Changes in fluorescence were used as an indicator of changes in [Ca²⁺]ᵢ and detected within cytosolic regions of interest. Changes in fluorescence were related to fluorescence at time 0s to give a fold increase equivalent to fluorescence/fluorescence at time 0s (F/F₀).

### Results

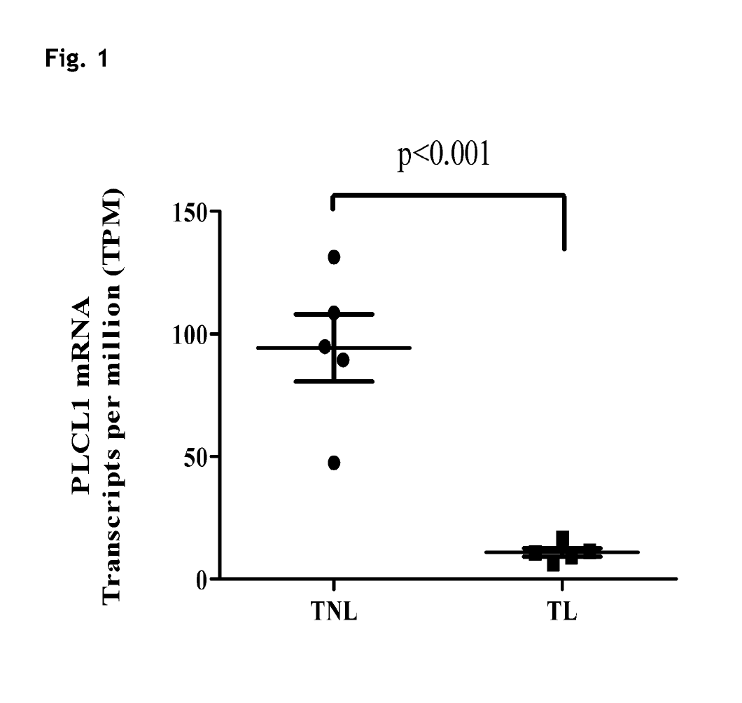

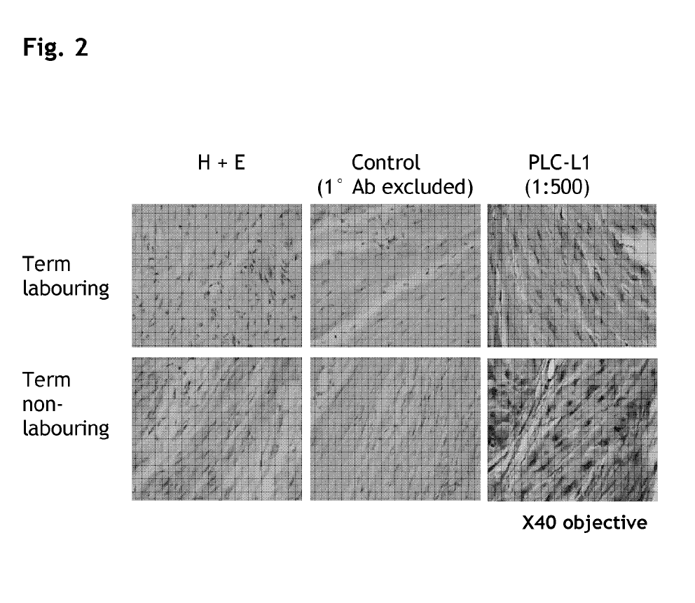

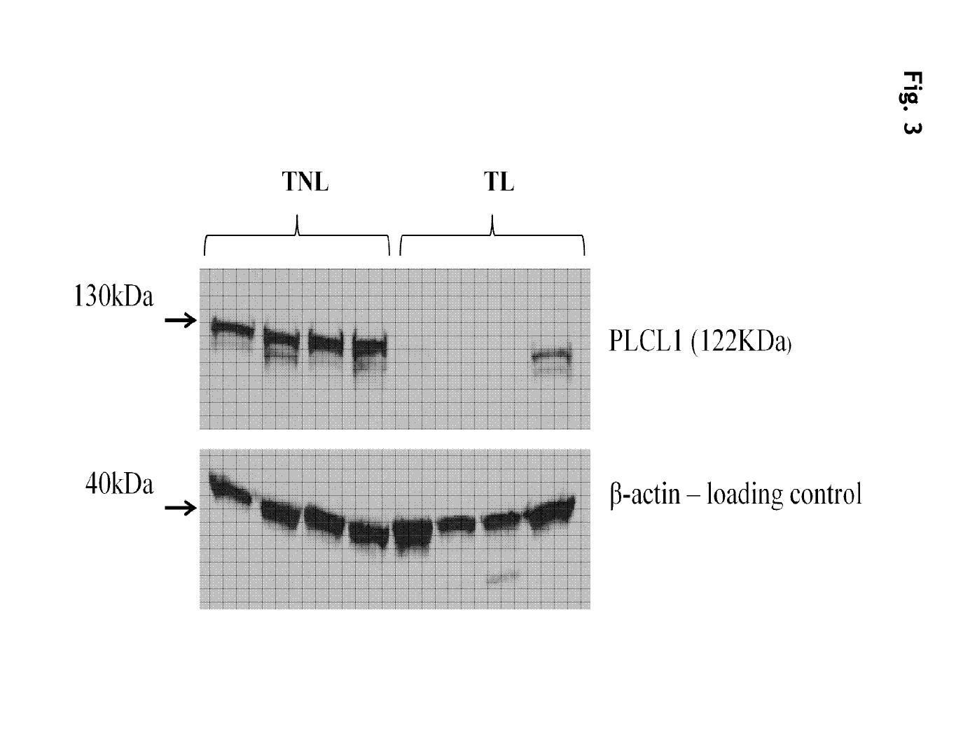

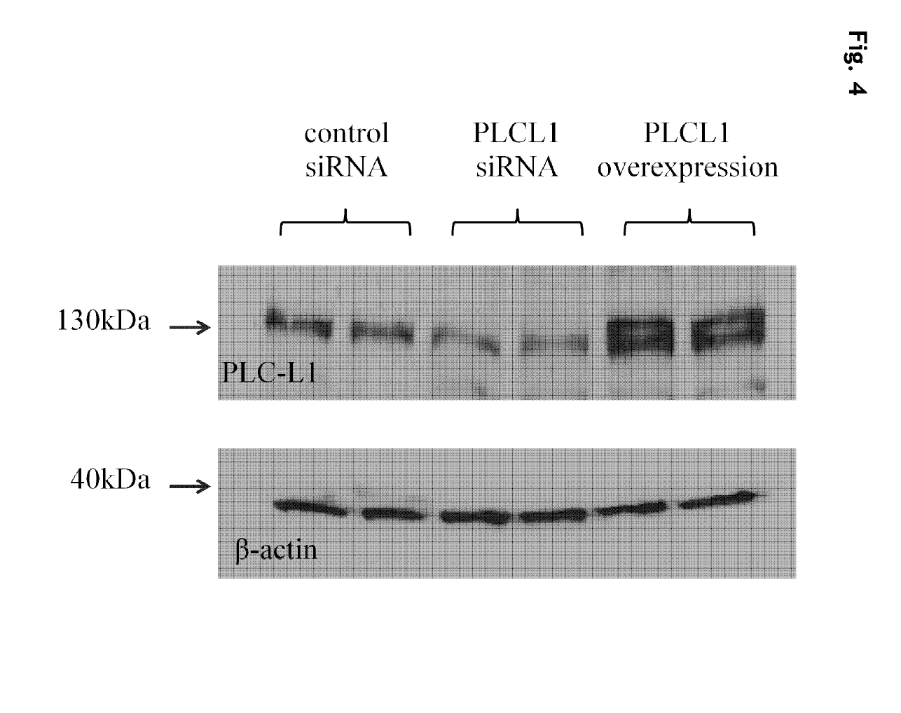

Laser capture micro-dissection was employed to isolate myometrial smooth muscle cells (MSM) from frozen samples of myometrium taken at caesarean section. High quality mRNA from these samples was isolated and then subjected to RNA-seq, which sequences all transcripts within a given population of mRNAs and provides an unbiased assessment of mRNA transcripts made in comparison to the population as a whole. Expression profiles were generated for 5 samples taken from patients not in labour (NIL) and compared with 5 samples taken from patients in spontaneous labour (LAB). From a comparison of gene lists genes which were significantly different between the patient groups and which were likely to modify cellular signalling during contractility were considered. In the top 10 differentially expressed genes comparing NIL vs LAB an outstanding candidate for modification of intracellular signalling was identified. This protein, phospholipase C-like 1 (PLC-L1) was significantly down-regulated (FIG. 1 P<0.0001) in samples taken from LAB patients compared with NIL patients. These changes were further confirmed at the protein level by immunohistochemistry (FIG. 2) and Western blotting (FIG. 3).

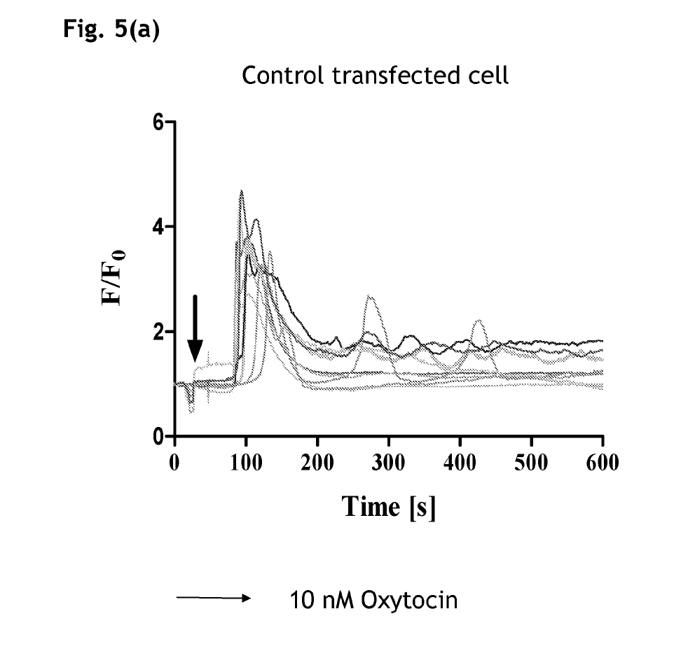

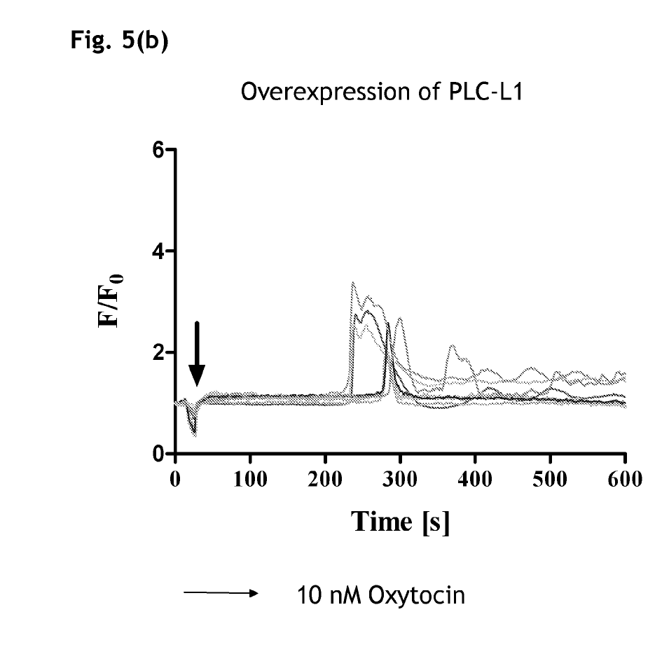

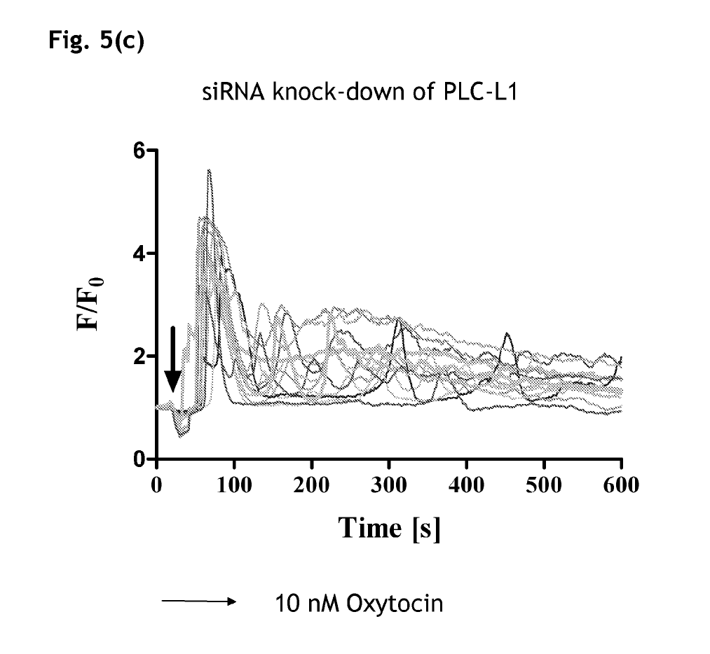

Endogenous expression of PLC-L1 was also overexpressed and knocked down in primary cultured human myometrium from not in labour patients (FIG. 4). In passage 1 cells, overexpression of PLC-L1 virtually eliminated any calcium transients when cells were challenged with 10 nM oxytocin (FIG. 5(b)). By contrast cells transfected with siRNA targeting PLC-L1 demonstrated greatly enhanced calcium oscillations (FIG. 5(c)) when compared to scrambled control (FIG. 5(a)). Thus in cultured human myometrial cells PLC-L1 appears to completely abrogate oxytocin mediated calcium signalling. If this were replicated in vivo the uterus would remain refractory to oxytocin, just as is observed in patients before term.

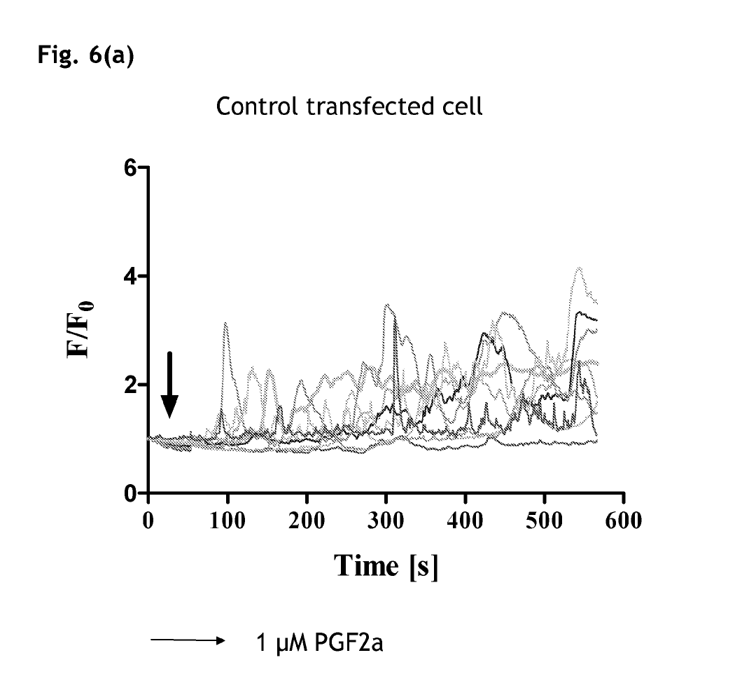

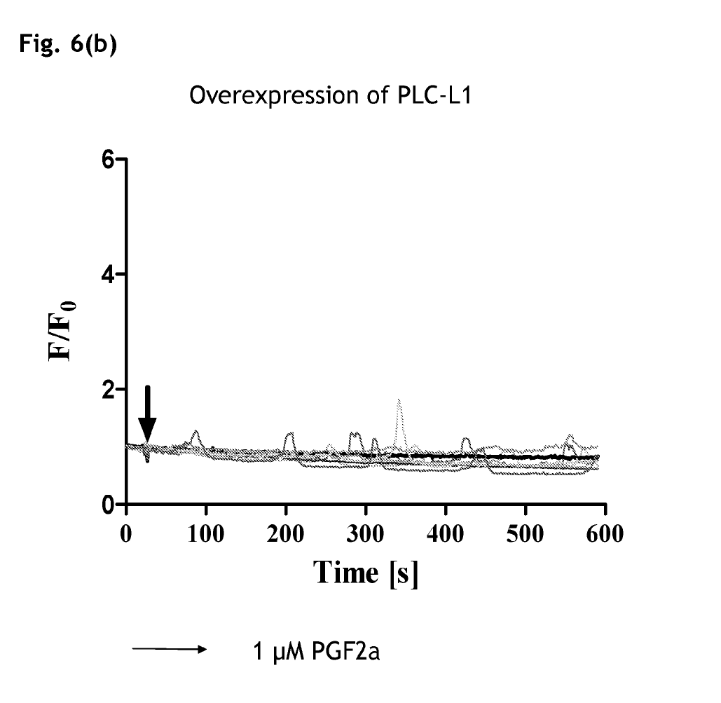

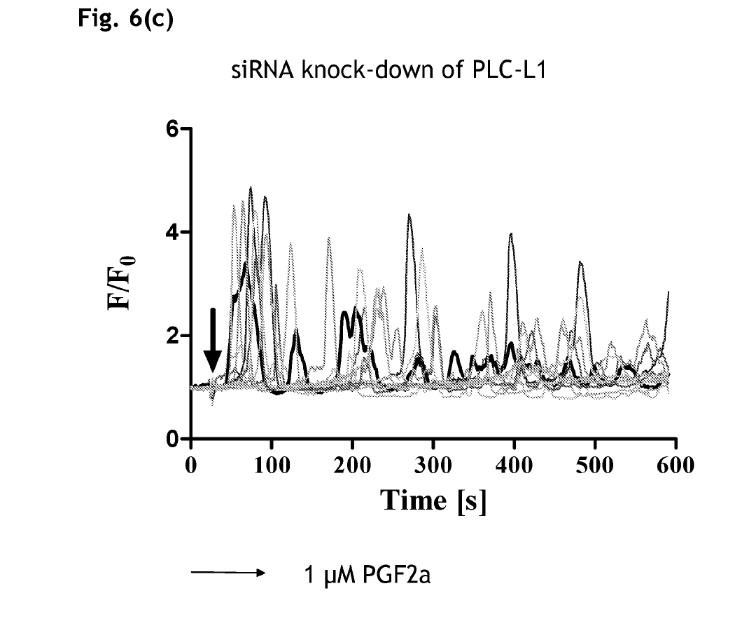

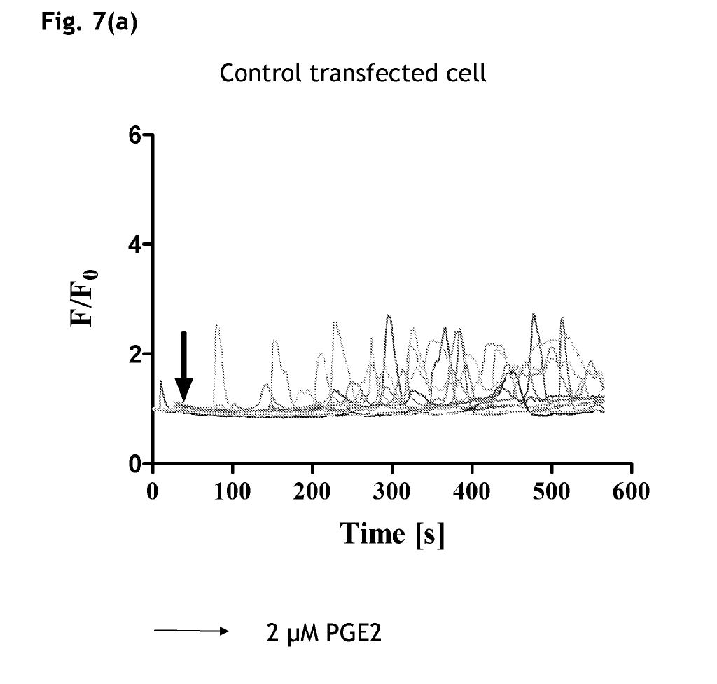

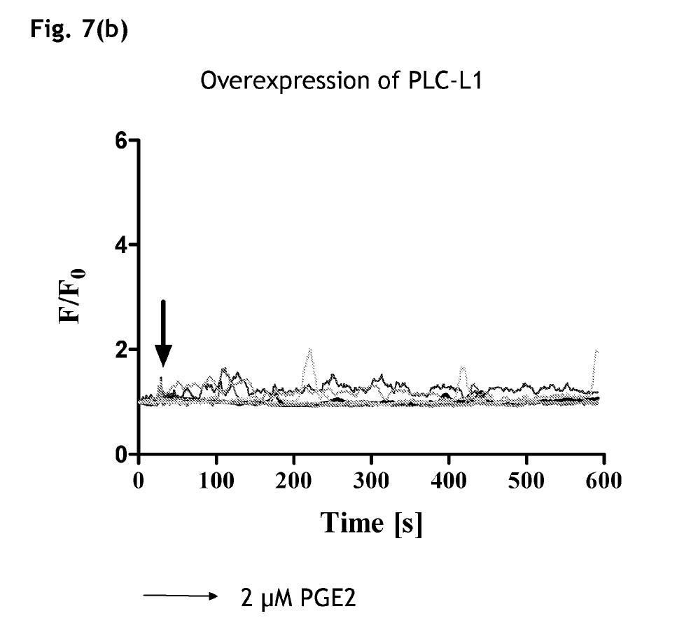

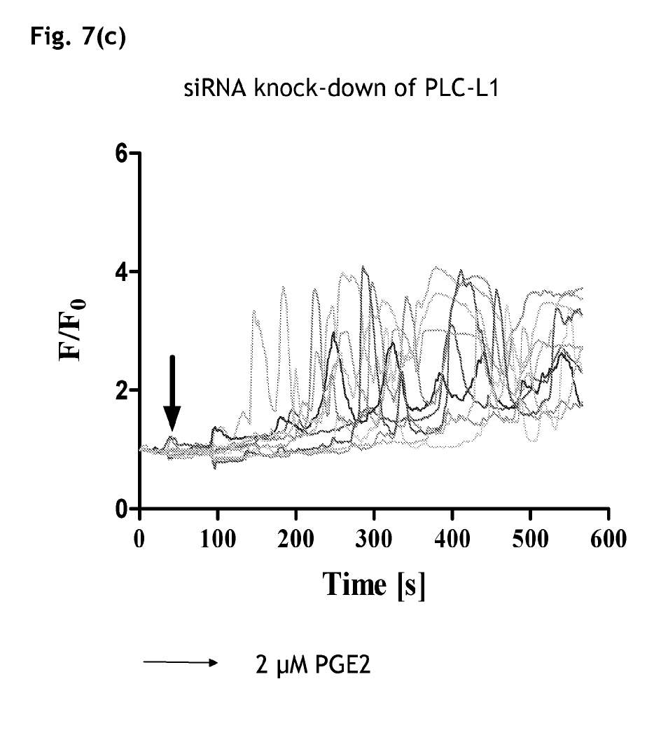

Comparable results are also shown following addition of PGF₂ₐ and PGE₂ (FIG. 6(a)-(c) and FIG. 7(a)-(c)).

These results suggest that during quiescence PLC-L1 binds to and sequesters IP₃ at the site of the ER. This effectively blocks G_{αq/11} signaling, irrespective of agonist, preventing the release of Ca²⁺ from cell stores and thus rendering the uterus insensitive to known stimulatory hormones. As labour approaches this break on signalling is removed and effective IP₃ signalling is restored.

1. A method of determining whether or not a patient is in labour, said method comprising measuring the amount of phospholipase C-like 1 (PLC-L1) in a sample from the patient and thereby determining whether or not the patient is in labour.

2. The method according to claim 1, wherein a decreased amount of PLC-L1 in the sample indicates that the patient is in labour.

3. The method according to claim 2, wherein said method comprises:
(a) measuring the amount of PLC-L1 in a sample from the patient; and
(b) comparing the amount of PLC-L1 in the sample with a control amount of PLC-L1,
wherein a decreased amount in the sample from the patient compared with the control amount indicates that the patient is in labour, and wherein an equivalent or increased amount in the sample from the patient compared with the control amount indicates that the patient is not in labour.

4. The method according to claim 3, wherein the amount in the sample from the patient which is decreased by at least 50% compared with the control amount indicates that the patient is in labour.

5. The method according to claim 3, wherein:
(a) the control amount is derived from comparable pregnant patients who are not in labour;
(b) the control amount is derived from the patient at an earlier stage in pregnancy;
(c) the control amount is derived from comparable pregnant patients who are not in labour and the amount in the sample from the patient which is decreased by at least 50% compared with the control amount indicates that the patient is in labour; or
(d) the control amount is derived from the patient at an earlier stage in pregnancy and the amount in the sample from the patient which is decreased by at least 50% compared with the control amount indicates that the patient is in labour.

6. (canceled)

7. The method according to claim 1, wherein the PLC-L1 is PLC-L1 mRNA or is PLC-L1 protein.

8. The method according to claim 3, wherein in step (a) the PLC-L1 is PLC-L1 mRNA and the control amount is from about 50 to 230 transcripts per million, where coverage per transcriptome base is 20-30, or in step (b) the PLC-L1 is PLC-L1 mRNA and the control amount is from about 50 to 230 transcripts per million, where coverage per transcriptome base is 20-30 and the amount in the sample from the patient is from about 5 to about 40 transcripts per million where coverage per transcriptome base is 20-30.

9. (canceled)

10. (canceled)

11. The method according to claim 1, wherein:
(a) the sample from the patient is urine, blood, serum, plasma, a cervical sample or a uterine sample;
(b) the method further comprises measuring the amount of oxytocin, prostaglandin F2_{α} and/or prostaglandin E₂ in a sample from the patient; or
(c) the patient is a human.

12. (canceled)

13. (canceled)

14. A kit for determining whether or not a patient is in labour, comprising a means for taking a sample from the patient and a reagent for measuring the amount of PLC-L1.

15. The kit according to claim 14, further comprising a reagent for measuring the amount of oxytocin, prostaglandin F2_{α} and/or prostaglandin E₂.

16. An oligonucleotide which specifically hybridises to (a) a part of SEQ ID NOs: 3 and/or 4 or (b) a variant with at least 90% homology to SEQ ID NOs: 3 and/or 4.

17. An oligonucleotide which comprises 50 or fewer consecutive nucleotides from (a) SEQ ID NOs: 1 and/or 2 or (b) a variant sequence with at least 90% homology to SEQ ID NOs: 1 and/or 2.

18. A method of inducing labour in a patient, said method comprising administering to the patient an inhibitor of PLC-L1 and thereby inducing labour in the patient.

19. The method according to claim 18, wherein the patient has been determined as not being in labour using the method of claim 1.

20. The method according to claim 18, said method comprising:
(a) measuring the amount of phospholipase C-like 1 (PLC-L1) in a sample from the patient;
(b) comparing the amount of PLC-L1 in the sample with a control amount of PLC-L1;
(c) if the amount in the sample from the patient is equivalent to or increased compared with the control amount administering the inhibitor of PLC-L1 to the patient and thereby inducing labour in the patient.

21. The method according to claim 18, wherein:
(a) the inhibitor is a small molecule inhibitor, an antibody, or an oligonucleotide which specifically hybridizes to (a) a part of SEQ ID NOs: 3 and/or 4 or (b) a variant with at least 90% homology to SEQ ID NOs: 3 and/or 4;
(b) the inhibitor is administered to the cervix or to the uterus of the patient; or
(c) the method further comprises administering to the patient oxytocin, prostaglandin F2_{α} and/or prostaglandin E₂.

22. (canceled)

23. (canceled)

24. A kit for inducing labour in a patient, said kit comprising an inhibitor of PLC-L1 and oxytocin, prostaglandin F2_{α} and/or prostaglandin E₂.

25. A method of preventing labour in a patient, said method comprising administering to the patient PLC-L1 and thereby preventing labour in the patient.

26. The method according to claim 25, wherein the patient has been determined as being in labour using the method of claim 1.

27. The method according to claim 25, said method comprising:
(a) measuring the amount of phospholipase C-like 1 (PLC-L1) in a sample from the patient;
(b) comparing the amount of PLC-L1 in the sample with a control amount of PLC-L1;
(c) if the amount in the sample from the patient is decreased compared with the control amount administering PLC-L1 to the patient and thereby preventing labour in the patient.

28. The method according to claim 25, wherein:
(a) the PLC-L1 comprises (a) the nucleotide sequence as show in SEQ ID NO: 1 or SEQ ID NO: 2, or variants thereof with at least 80% homology to SEQ ID NO: 1 or SEQ ID NO: 2 or (b) the amino acid sequence as shown in SEQ ID NO:5 or SEQ ID NO: 6, or variants thereof with at least 80% homology to SEQ ID NO:5 or SEQ ID NO: 6;
(b) the PLC-L1 is administered to the cervix or to the uterus of the patient; or
(c) the method further comprises administering to the patient an inihibtor of oxytocin, prostaglandin F2_{α}, prostaglandin E₂ or Atosiban, Terbutaline, Ritodrine and/or Nifedipine.

29. (canceled)

30. (canceled)

31. A kit for preventing labour in a patient, said kit comprising PLC-L1 and an inihibtor of oxytocin, prostaglandin F2_{α} prostaglandin E₂, or Atosiban, Terbutaline, Ritodrine and/or Nifedipine.
